SEQUENCE LISTING (103 sequences)

SEQ ID NO: 1 - protein, 441 aa, Homo sapiens
EVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 2 - protein, 215 aa, Homo sapiens
ESALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQGQVTVACEEGWTLTGCSALPGTSHVLGAYAVDNTCVVRSRDVSTTGSTSEEAVTAVAICCRSRHLAQASQELQ

SEQ ID NO: 4 - protein, 14 aa, Homo sapiens
TGTSSDVGGYNSVS

SEQ ID NO: 5 - protein, 7 aa, Homo sapiens
EVSNRPS

SEQ ID NO: 6 - protein, 9 aa, Homo sapiens
NSYTSTSMV

SEQ ID NO: 7 - protein, 10 aa, Homo sapiens
GYTLTSYGIS

SEQ ID NO: 8 - protein, 16 aa, Homo sapiens
WVSFYNGNTNYAQKLQ

SEQ ID NO: 9 - protein, 6 aa, Homo sapiens
GYGMDV

SEQ ID NO: 10 - DNA, 345 bp, Homo sapiens
  source 1..345: mol_type = other DNA
caggttcagctggtgcagtctggagctgaggtgaagaagcctggggcctcagtgaaggtctcctgcaaggcttctggttacaccttaaccagctatggtatcagctgggtgcgacaggcccctggacaagggcttgagtggatgggatgggtcagtttttataatggtaacacaaactatgcacagaagctccagggcagaggcaccatgaccacagacccatccacgagcacagcctacatggagctgaggagcctgagatctgacgacacggccgtgtattactgtgcgagaggctacggtatggacgtctggggccaagggaccacggtcaccgtctcctct

SEQ ID NO: 11 - DNA, 327 bp, Homo sapiens
  source 1..327: mol_type = other DNA
cagtctgccctgactcagcctgcctccgtgtctgggtctcctggacagtcgatcaccatctcctgcactggaaccagcagtgacgttggtggttataactctgtctcctggtaccaacagcacccaggcaaagcccccaaactcatgatttatgaggtcagtaatcggccctcaggggtttctaatcgcttctctggctccaagtctggcaacacggcctccctgaccatctctgggctccaggctgaggacgaggctgattattactgcaattcatatacaagcaccagcatggtattcggcggagggaccaagctgaccgtccta

SEQ ID NO: 12 - DNA, 1380 bp, Homo sapiens
  source 1..1380: mol_type = other DNA
atggactggacctggaggatccttttcttggtggcagcagccacaggtgtccactccgaggttcagctggtgcagtctggagctgaggtgaagaagcctggggcctcagtgaaggtctcctgcaaggcttctggttacaccttaaccagctatggtatcagctgggtgcgacaggcccctggacaagggcttgagtggatgggatgggtcagtttttataatggtaacacaaactatgcacagaagctccagggcagaggcaccatgaccacagacccatccacgagcacagcctacatggagctgaggagcctgagatctgacgacacggccgtgtattactgtgcgagaggctacggtatggacgtctggggccaagggaccacggtcaccgtctcctctgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 13 - DNA, 711 bp, Homo sapiens
  source 1..711: mol_type = other DNA
atggacatgagggtgcccgctcagctcctggggctcctgctgctgtggctgagaggtgccagatgtgagtctgccctgactcagcctgcctccgtgtctgggtctcctggacagtcgatcaccatctcctgcactggaaccagcagtgacgttggtggttataactctgtctcctggtaccaacagcacccaggcaaagcccccaaactcatgatttatgaggtcagtaatcggccctcaggggtttctaatcgcttctctggctccaagtctggcaacacggcctccctgaccatctctgggctccaggctgaggacgaggctgattattactgcaattcatatacaagcaccagcatggtattcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 14 - protein, 115 aa, Homo sapiens
QVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSS

SEQ ID NO: 15 - protein, 109 aa, Homo sapiens
QSALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVL

SEQ ID NO: 16 - protein, 115 aa, Homo sapiens; identical to SEQ ID NO: 14

SEQ ID NO: 17 - protein, 109 aa, Homo sapiens; identical to SEQ ID NO: 15

SEQ ID NO: 18 - protein, 239 aa, Homo sapiens
MRLPAQLLGLLMLWVPGSSGDIVMTQTPLSSPVTLGQPASISCRSSQSLVHSDGNTYLNWLQQRPGQPPRLLIYKISNRFSGVPDRFTGSGAGTDFTLKISRVEAEDVGVYTCMQVTQFPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 470 aa, Homo sapiens [sequence omitted]

SEQ ID NO: 20 - protein, 235 aa, Homo sapiens
METPAQLLFLLLLWLPDTTGEIVLTQSPGTLSLSPGDRATLSCRASQSVSSSYFAWYQQKPGQAPRLLIYGASSRATGIPDRFSGSGSGTDFTLTISRLEPEDFAVYYCQQYDRSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 463 aa, Homo sapiens
MDWTWRILFLVAAATGAHSQVQLVQSGAEVKKPGASVKVSCKSSGYTFTGYYMHWVRQAPGQGLEWMGWINPNSGGTNYAQKFKGRVTMTRDTSISTAYMELSRLRSDDTAVYYCARDKWLDGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 22 - protein, 235 aa, Homo sapiens
MAWALLLLTLLTQDTGSWAQSVLTQSPSASGTPGQRVTISCSGSSSNIGSNYVYWYQQLPGAAPKLLILRNNQRPSGVPDRFSGSKSGTSASLTISGLRSEDEADYYCAAWDDSLSGWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 23 - protein, 479 aa, Homo sapiens
MDMRVPAQLLGLLLLWLRGARCEVQLVESGGGLVKPGGSLRLSCAASGFTFSNAWMSWVRQAPGKGLEWVGRIKSKTDGGTTDYTAPVKGRFTISRDDSKNTLYLQMNSLKAEDTAVYYCTTDRTGYSISWSSYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 24 - protein, 235 aa, Homo sapiens
METPAQLLFLLLLWLPDTTGEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 467 aa, Homo sapiens
MEFGLSWLFLVAILKGVQCEVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 26 - protein, 242 aa, Homo sapiens
MDMRVPAQLLGLLLLWLRGARCDIVMTQTPLSLPVTPGEPASISCRSSQSLLNSVDGSTNLDWYLQKPGQSPQLLIYTLSYRASGVPDRFSGSGSGTDFTLKISRVEAEDVGVYYCMQRIEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 465 aa, Homo sapiens
MELGLCWVFLVAILEGVQCEVQLVESGGGLVQPGGSLRLSCAASGFTFSSYSMNWVRQAPGKGLEWVSYISSSGSSIYYADSVKGRFTISRDNAKNSLYLQMNSLRDEDTAVYYCARERYYGDTPFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 28 - protein, 233 aa, Homo sapiens
MSPSQLIGFLLLWVPASRGEIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 463 aa, Homo sapiens
MGSTAILALLLAVLQGVCAEVQLMQSGAEVKKPGESLKISCKGSGYSFSFHWIAWVRQMPGKGLEWMGIIHPGASDTRYSPSFQGQVTISADNSNSATYLQWSSLKASDTAMYFCARQRELDYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - protein, 236 aa, Homo sapiens
MDMRVPAQLLGLLLLWLRGARCDIQMTQSPSSLSASVGDRVTITCKASQDINKYVAWYQQKPGKAPKLLIYYTSWLQPGVPSRFSGSGSGTDFTFTISSLQPEDIATYYCLQYDNLLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 467 aa, Homo sapiens
MDMRVPAQLLGLLLLWLRGARCEVQLVESGGGLVQPGGSLRLSCAASGFTFSRYWMNWVRQAPGKGLEWVAQIRLKSDNYATHYAESVKGRFTISRDNAKNSLYLQMNSLRAEDTAVYYCTEGLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 236 aa, Homo sapiens
MDMRVLAQLLGLLLLCFPGARCDIQMTQSPSSLSASVGDRVTITCRASQGISNWLAWYQQKPEKAPKSLIYAASSLQSGVPSRFSGSGSGTDFTLTISSLQPEDFATYYCQQYDSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 466 aa, Homo sapiens
MELGLNWVFLVAILEGVHCEVQLVESGGGLVQPGGSLRLSCAASGFTFSSYWMSWVRQAPGKGLEWVAYIKQDGNEKYYVDSVKGRFTISRDNAKNSLYLQMNSLRAEDTAVYYCAREGILWFGDLPTFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 34 - protein, 241 aa, Homo sapiens
MDMRLPAQLLGLLMLWVPGSSGDVLMTQSPLSLPVTLGQPASISCRSSQSIVHSNGNTYLEWYLQRPGQSPKLLIYKVSNRFSGVPDRFSGSGSGTDFTLKISRVEAEDVGVYYCFQGSHVPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 470 aa, Homo sapiens
MDTLCSTLLLLTIPSWVLSQVTLKESGPALVKPTQTLTLTCTFSGFSLRTSGMGVGWIRQPPGKALEWLAHIWWDDDKSYNPSLKSQLTISKDTSKNQVVLTMTNMDPVDTATYYCARRNYYYDDYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 235 aa, Homo sapiens
METPAQLLFLLLLWLPDTTGEIVLTQSPGTLSLSPGERATLSCRASQSVSSSYFAWYQQKPGQAPRLLIYGTSSRATGIPDRFSGSGSGTDFTLTVSRLEPEDFAVYYCQQYDRSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 463 aa, Homo sapiens
MDWTWRILFLVAAATGAHSQVQLVQSGAAVKKPGASVKVSCKASGYTFTGYYIHWVRQAPGQGLEWMGWINPNSGGTNYAQKFQGRVTMTRDTSISTASMELSRLRSDDTAVYFCARDRWLDAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 38 - protein, 235 aa, Homo sapiens
METPAQLLFLLLLWLPDTTGEIVLTQSPGTLSLSPGERATLSCRASQSVSSGYLTWYQQKPGQAPRLLIYGASSRATGIPDRFSGSGSGTDFTLTISRLEPEDFAVYYCQQYGNSLSRFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 474 aa, Homo sapiens
MDMRVPAQLLGLLLLWLRGARCQVQLVESGGGVVQPGRSLRLSCAASGFTFSSYGMHWVRQAPGKGLEWVAVIWYDGSNKYYADSVKGRFIISRDKSKNTLYLQMNSLRAEDTAVYYCARAGGIAAAGLYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 40 - protein, 236 aa, Homo sapiens
MDMRVPAQLLGLLLLWLRGARCSSELTQDPTVSVALGQTVKITCQGDSLRSFYASWYQQKPGQAPVLVFYGKNNRPSGIPDRFSGSSSGNTASLTITGAQAEDEADYYCNSRDSSVYHLVLGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 41 - protein, 478 aa, Homo sapiens
MDMRVPAQLLGLLLLWLRGARCQVQLVQSGAEVKKPGASVKVSCKASGYTFTGYYMHWVRQAPGQGLEWMGWINPNSGGTNYAQKFQGRVTMTRDTSISTAYMELSRLRSDDTAVYFCARDQMSIIMLRGVFPPYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 42 - protein, 241 aa, Homo sapiens
MDMRVPAQLLGLLLLWLRGARCDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 475 aa, Homo sapiens
MDMRVPAQLLGLLLLWLRGARCEVQLVESGGGLVQPGGSLRLSCAASGFTFSSYWMSWVRQAPGKGLEWVASIKQDGSEKYYVDSVKGRFTISRDNARNSLYLQMNSLRAEDTAVYYCARDLVLMVYDIDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 44 - protein, 241 aa, Homo sapiens
MDMRVPAQLLGLLLLWLRGARCDIVMTQSPLSLPVTPGEPASISCRSSQSLLHSNGYNYLDWYLQKPGQSPQLLIYLGSNRASGVPDRFSGSGSGTHLTLKISRVEAEDVGVYYCMQTLQTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 475 aa, Homo sapiens
MDMRVPAQLLGLLLLWLRGARCEVQLVESGGGLVQPGGSLRLSCAASGFTFSNYWMSWVRQAPGKGLEWVASIKQDGSEKYYVDSVKGRFAISRDNAKNSLFLQMNSLRAEDTAVYYCARDLVLMVYDIDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 46 - protein, 235 aa, Homo sapiens
MTCSPLLLTLLIHCTGSWAQSVLTQPPSVSAAPGQKVTISCSGSSSNIGNNYVSWYQQLPGTAPKLLIYDNNKRPSGIPDRFSGSKSGTSTTLGITGLQTGDEADYYCGTWDSRLSAVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 47 - protein, 475 aa, Homo sapiens
MEFGLSWVFLVALLRGVQCQVQLVESGGGVVQPGRSLRLSCAASGFTFSSFGMHWVRQAPGKGLEWVAVISFDGSIKYSVDSVKGRFTISRDNSKNTLFLQMNSLRAEDTAVYYCARDRLNYYDSSGYYHYKYYGMAVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 48 - protein, 234 aa, Homo sapiens
MDMRVPAQLLGLLLLWLRGARCYELTQPPSVSVSPGQTATITCSGDKLGERYASWYQQRPGQSPVLVIYQDIKRPSGIPERFSGSNSGNTATLTISGTQAMDEADYFCQAWYSSTNVLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 49 - protein, 466 aa, Homo sapiens
MEFGLSWVFLVALLRGVQCQVQLVESGGGVVQPGRSLRLSCAASGFTFSSYGMHWVRQAPGKGLEWVAVIWYAESNKYYADSVKGRFTISRDNSKNTLYLQMNSLRAEDTAVYYCARAQEGIAPDAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 50 - protein, 234 aa, Homo sapiens
MGSTAILGLLLAVLQGGRAEIVLTQSPGTLSLSPGERATLSCRASQSVSSSYLAWHQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 463 aa, Homo sapiens
MGSTAILGLLLAVLQGGRAEVQLLESGGGLVQPGGSLRLSCAASGFTFSTYVMSWVRQAPGKGLEWVSSISGSGLGSYYADSVKGRFTISRDNSKNTLYLQMNSLRAEDTAVYYCAKEAHRGPFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 52 - protein, 239 aa, Homo sapiens
MRLLAQLLGLLMLWVPGSSGDIVMTQTPLSSPVTLGQPASISCRSSQSLVHSDGNTYLSWLQQRPGQPPRLLIYKKFNRFSGVPDRFSGSGAGTDFTLKISRVEAEDVGVYYCMQATQIPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 472 aa, Homo sapiens
MEFGLSWVFLVALLRGVQCQVQLVESGGGVVQPGRSLRLSCAASGFTFSFYGMHWVRQAPGKGLEWVAVIWYDGSNKYYADSVKGRFTISRDNSKNTLYLQMNSLRAEDTAVYYCARGGYDYVWGSYRRNSDFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 54 - protein, 235 aa, Homo sapiens
MEAPAQLLFLLLLWLPDTTGEIVMTQSPATLSVSPGERATLSCRASQSVDSNLAWYRQKPGQAPRLLIYGASTRATGIPARFSGSGSGTEFTLTISSLQSEDFAVYYCQQYINWPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 465 aa, Homo sapiens
MKHLWFFLLLVAAPRWVLSQVQLQESGPGLVKPSETLSLTCTVSGGSISIYYWSWIRQPPGKGLEWIGYVYYSGSTNYNPSLKSRVTISVDTSKNQFSLKLNSVTAADTAVYYCARGGYDFWSGYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 56 - protein, 240 aa, Homo sapiens
MVLQTQVFISLLLWISGAYGDIVLTQSPDSLAVSLGERATINCKSSQSILYSSSNENFLTWYQQKPGQPPKLLIYWASTRESGVPDRFSGSGSGTDFTLTISSLQPEDVAVYYCQQYFSVFRTFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 465 aa, Homo sapiens
MDWTWRILFLVAAATGAHSQVQLVQSGAEVKKPGASVKVSCKASGYTFTGYYIHWVRQAPGQGLEWMGWINPNSGGTNSAQKFRGRVTMTRDTSISTAYMELSRLRSDDTAVYYCAREGGYSYGYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 58 - protein, 236 aa, Homo sapiens
MDMRVPAQLLGLLLLWFPGARCDIQMTQSPSSLSASVGDRVTITCRASQGIRNDLGWYQQKPGKAPKRLIYAASSLQSGVPSRFSGSGSGTEFTLTISSVQPEDFVTYYCLQHNSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 473 aa, Homo sapiens
MEFGLSWVFLVALLRGVQCQVQLVESGGGVVQPGRSLRLSCAASGFTFSSYGMHWVRQAPGKGLEWVAVMWYDGSNKDYVDSVKGRFTISRDNSKNTLYLQMNRLRAEDTAVYYCAREKDHYDILTGYNYYYGLDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 60 - protein, 235 aa, Homo sapiens
METPAQLLFLLLLWLPDTTGEIVLTQSPGTLSLSPGERATLSCRASQYISNTYLAWFQQKPGQAPRLLIYGAATRATGIPDRFSGSGSGTDFTFTISRLEPEDFAVYYCQQYGSSPWTFGQGTTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 463 aa, Homo sapiens
MDWTWRILFLVAAATGAHSQVQLVQSGAEVKKPGASVKVSCKASGYTFTGYYMHWVRQAPGQGLEWMGWINPNSGGTNYAQRFRGRVTMTRDTSISTAYMELSRLRSDDTAVYYCARAPYDWTFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 62 - protein, 236 aa, Homo sapiens
MDMRVPAQLLGLLLLWLRGARCDIQMTQSPSSVSASVGDRVTITCRASQGISNWLAWYQQKPGTAPKLLIYAASSLQSGVPSRFSGSGSGTDFTLTISSLQPEDFATYYCQQANSFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 469 aa, Homo sapiens
MDMRVPAQLLGLLLLWLRGARCQVQLVQSGTEVKKPGASMKVSCKASGYTFTSYYMHWVRQAPGQGLEWMGIINPSGDSTSYAQKFQGRVTMTRDTSTNTVYMELSSLRSEDTAMYYCARDVEVRGISHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 64 - protein, 238 aa, Homo sapiens
MGWSCIILFLVATATGVHSDIQLTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGGGTKVQIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 465 aa, Homo sapiens
MGWSCIILFLVATATGVHSQVQLVQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 235 aa, Homo sapiens
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 466 aa, Homo sapiens
MGSTAILALLLAVLQGVCAEVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 241 aa, Homo sapiens
MDMRVPAQLLGLLLLWLRGARCDIVMTQSPLSLPVTPGEPASISCRSSQSLLHSHGYNYLDWYLQKPGQSPQLLIYLGSNRASGVPDRFSGSGSGTDFTLKISRVEAEDVGVYYCMQGTHWPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 470 aa, Homo sapiens
MDMRVPAQLLGLLLLWLRGARCEVQLVQSGGGVVQPGRSLRLSCAASGFTFSSYGMHWVRQAPGKGLEWVSYISSSGSTIYYADSVKGRFTISRDNAKNSLYLQMNSLRAEDTAVYYCARDLLDYDLLTGYGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 70 - protein, 235 aa, Homo sapiens
METPAQLLFLLLLWLPDTTGEIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 471 aa, Homo sapiens
MKHLWFFLLLVAAPRWVLSQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 236 aa, Homo sapiens
MDMRVPAQLLGLLLLWLSGARCDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 464 aa, Homo sapiens
MKHLWFFLLLVAAPRWVLSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 74 - protein, 236 aa, Homo sapiens
METPAQLLFLLLLWLPDTASEIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 460 aa, Homo sapiens
MGSTAILGLLLAVLQGVASEVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 76 - protein, 234 aa, Homo sapiens
METPAQLLFLLLLWLPDTTGEIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 467 aa, Homo sapiens
MGSTAILALLLAVLQGVCAEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 241 aa, Homo sapiens
MDMRVPAQLLGLLLLWLRGARCDVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 471 aa, Homo sapiens
MDMRVPAQLLGLLLLWLRGARCQVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARWTGRTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 234 aa, Homo sapiens
MEAPAQLLFLLLLWLPDTTGEIVMTQSPATLSVSPGERATLSCRASQSVSSNLAWFQQKPGQAPRPLIYDASTRATGVPARFSGSGSGTDFTLTISSLQSEDFAVYYCQQYDNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 461 aa, Homo sapiens
MEWTWRVLFLVAAATGAHSQVQLVQSGAEVKKPGASVKVSCKASGYTFTRYGISWVRQAPGQGLEWMGWISTYSGNTNYAQKLQGRVTMTTDTSTSTAYMELRSLRSDDTAVYYCARRQLYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 82 - protein, 239 aa, Homo sapiens
MRLPAQLLGLLMLWIPGSSADIVMTQTPLSLSVTPGQPASISCKSGQSLLHSDGKTYLYWYLQKPGQPPQFLIYEVSNRFSRVPDRFSGSGSGTDFTLRISRVEAEDVGIYYCMQSIQLPWTFGQGTQVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 459 aa, Homo sapiens
MEFGLSWVFLVALLRGVQCQVQLVESGGGVVQPGRSLRLSCAASGFTFSGYGMHWVRQAPGKGLEWVAVISYDGNDKYYADSVKGRFTISRDNAKNTLYLQMNSLRAEDTAVYYCARELRVLWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 84 - protein, 235 aa, Homo sapiens
MDMRVPAQLLGLLLLWLRGARCDIQMTQSPSSLSASVGDRVTITCRASQDISSYLNWYQQKPGKAPKLLIYSTSRLNSGVPSRFSGSGSGTDFTLTISSLQPEDFATYYCQQDIKHPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 465 aa, Homo sapiens
MDWTWRILFLVAAATGAHSEVQLVQSGAEVKKPGSSVKVSCKASGFTFTDYIMHWVRQAPGQGLEWMGYINPYNDDTEYNEKFKGRVTITADKSTSTAYMELSSLRSEDTAVYYCARSIYYYDAPFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 86 - protein, 233 aa, Homo sapiens
MGSTAILGLLLAVLQGGRASYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 87 - protein, 467 aa, Homo sapiens
MGSTAILGLLLAVLQGGRAQMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 88 - protein, 234 aa, Homo sapiens
MGVPTHLLGLLLLWITHAICDIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 89 - protein, 465 aa, Homo sapiens
MDIRLSLAFLVLFIKGVQCEVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSAETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 90 - protein, 237 aa, Homo sapiens
MDMRVPAQLLGLLLLWLRGARCESALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 91 - protein, 460 aa, Homo sapiens
MDWTWRILFLVAAATGVHSEVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 92 - protein, 234 aa, Homo sapiens
METPAQLLFLLLLWLPDTTGEIVMTQSPATLSVSPGERATLSCRASQSVSSNLAWYQQKPGQAPRLLIYGAATRATGIPARVSGSGSGTEFTLTISSLQSEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 472 aa, Homo sapiens
MDMRVPAQLLGLLLLWLRGARCQVQLVESGGGVVQPGRSLRLSCAASGFTFSNYGMHWVRQAPGEGLEWVAAIWFDASDKYYADAVKGRFTISRDNSKNTLYLQMNSLRAEDTAVYYCARDQAIFGVVPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 231 aa, Homo sapiens
MAWIPLFLGVLAYCTGSVASYEVTQAPSVSVSPGQTASITCSGDKLGDKYACWYQQKPGQSPVLVIYQDSKRPSGIPERFSGSNSGNTATLTISGTQAMDEADYYCQAWDSSTAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 95 - protein, 467 aa, Homo sapiens
MDWTWSILFLVAAATGAHSQVQLVQSGAEVKKPGASVKVSCKASGYTFTSYGLSWVRQAPGQGLEWMGWIIPYNGNTNSAQKLQGRVTMTTDTSTSTAYMELRSLRSDDTAVYFCARDRDYGVNYDAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 96 - protein, 235 aa, Homo sapiens
MAWAPLLLTLLAHCTGSWANFMLTQPHSVSESPGKTVAISCTRNSGSIASNSVQWYQQRPGSSPTTVIFEDNQRPSGVPDRFSGSIDSSSNSASLTISGLKTEDEADYYCQSYDSNNWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 97 - protein, 470 aa, Homo sapiens
MEFGLSWVFLVALLRGVQCQVQLVESGGGVVQPGRSLRLSCAASGFTFSSYVMHWVRQAPGKGLEWVAVIWYDGSNKYYADSVKGRFTISRDNSKNTLYLQMNSLRAEDTAVYYCAREGYDYGEDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 98 - protein, 236 aa, Homo sapiens
MDMRVPAQLLGLLLLWLRGARCDIQMTQSPSSLSASVGDRVTITCRASQDISNYLNWYQQKPGKAPKLLIYYTSRLLSGVPSRFSGSGSGTDFTLTISSLQPEDFATYYCQQGDTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 468 aa, Homo sapiens
MDWTWRILFLVAAATGAHSEVQLVQSGAEVKKPGASVKVSCKASGYTFTDYNMHWVRQAPGQGLEWMGEINPNSGGAGYNQKFKGRVTMTTDTSTSTAYMELRSLRSDDTAVYYCARLGYDDIYDDWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 100 - protein, 240 aa, Homo sapiens
MVLQTQVFISLLLWISGAYGDIVMTQSPDSLAVSLGERATINCKSSQSVLDSSDNKNYLAWYQQKPGQPPKLLIYWASNRESGVPDRFSGSGSGTDFTLTISSLQAEDVAVYYCQQYYSDPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 465 aa, Homo sapiens
MDWTWSILFLVAAPTGAHSQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESWFGEVFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 102 - protein, 236 aa, Homo sapiens
MDMRVPAQLLGLLLLWLRGARCDIQMTQSPSSVSASVGDRVTITCRASQGISSWLAWYQQKPGKAPKLLIYGASNLESGVPSRFSGSGSGTDFTLTISSLQPEDFANYYCQQANSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 466 aa, Homo sapiens
MDMRVPAQLLGLLLLWLRGARCQVQLVQSGAEVKKPGASVKVSCKVSGYTLSDLSIHWVRQAPGKGLEWMGGFDPQDGETIYAQKFQGRVTMTEDTSTDTAYMELSSLKSEDTAVYYCATGSSSSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

(19) United States
(12) Patent Application Publication
(10) Pub. No.: US 2025/0367115 A1
(43) Pub. Date: Dec. 4, 2025

(54) Excipients To Reduce the Viscosity of Antibody Formulations and Formulation Compositions

(71) Applicant: AMGEN INC., Thousand Oaks, CA (US)

(72) Inventors: Jian Hua GU, Thousand Oaks, CA (US); Robert M. FESINMEYER, Newbury Park, CA (US); Rulin QIAN, Newbury Park, CA (US)

(73) Assignee: AMGEN INC., Thousand Oaks, CA (US)

(21) Appl. No.: 19/025,406

(22) Filed: Jan. 16, 2025

Related U.S. Application Data
(62) Division of application No. 16/609,448, filed on Oct. 29, 2019, now Pat. No. 12,257,343.
(60) Provisional application No. 62/492,049, filed on Apr. 28, 2017.

(51) Int. Cl. A61K 9/08; A61K 9/19; A61K 47/18; C07K 16/24; C07K 16/40

(52) U.S. Cl. CPC A61K 9/08; A61K 9/19; A61K 47/183; C07K 16/244; C07K 16/40

(57) ABSTRACT

This invention concerns a series of excipients capable of effectively reducing the viscosity of protein formulations. The viscosity reducing excipients are oligopeptides (e.g., dipeptides, tripeptides) comprising at least one arginine. The peptides may also include basic or acidic or hydrophilic or hydrophobic/aromatic amino acids. An arginine residue may be either at the amino or the carboxyl end of the oligopeptide.

### FIELD OF THE INVENTION

This invention relates to biopharmaceuticals, particularly to therapeutic antigen binding proteins, methods of use thereof, pharmaceutical formulations thereof, and processes of making pharmaceutical formulations. In particular, this invention relates to excipients in pharmaceutical formulations to reduce viscosity.

### BRIEF DESCRIPTION OF THE SEQUENCE LISTING

Incorporated herein by reference in its entirety is a Sequence Listing entitled, “A-2111-US—PSP_SeqList_ST25.txt”, comprising SEQ ID NO:1 through SEQ ID NO:103, which includes nucleic acid and/or amino acid sequences disclosed herein. The Sequence Listing has been submitted herein in ASCII text format via EFS, and thus constitutes both the paper and computer readable form thereof. The Sequence Listing was first created using PatentIn on Apr. 27, 2017, and is 278 KB in size.

### BACKGROUND OF THE INVENTION

The formulation of pharmaceutical proteins, polypeptides, and other biopharmaceuticals can be challenging. Oral formulation of pharmaceutical proteins is typically unsuitable because they are degraded by the digestive process. Transdermal administration is also generally unsuitable for proteins, because they are too large to pass through the skin effectively. As for pulmonary formulations, only one insulin product has been introduced to the market with limited success.

Pharmaceutical proteins are therefore typically administered by injection, but there are problems in formulating proteins for injection, as well. In conventional solutions, proteins are generally unstable. They are prone to degradation, such as deamidation, aggregation and precipitation, from both chemical and physical processes. Aggregation, precipitation, and viscosity are particularly problematic for most proteins, especially at high protein concentrations. Lyophilized proteins are generally more stable than proteins in solution, but the concomitant inconvenience affects patient compliance.

Developing protein formulations is particularly challenging at high concentrations. It has been reported that a variety of proteins cannot be stably formulated at high concentrations in solution (U.S. Pat. No. 9,364,542). Some formulations with proteins at relatively high concentrations are not stable, resulting in aggregation or precipitation.

Another major challenge in the development of high concentration protein formulations is viscosity, which is a critical input for drug delivery, device design and manufacturing. High viscosity formulations are difficult to handle during manufacturing, including at the bulk and filling stages. High viscosity formulations are also difficult to draw into a syringe and inject, making administration to the patient difficult and unpleasant. The need to identify compounds that are useful for reducing viscosity of highly concentrated protein formulations, to develop methods of reducing the viscosity of such formulations, and to provide pharmaceutical formulations with reduced viscosity exists throughout the pharmaceutical industry. Many proteins suffer from sub-optimal formulations or cannot be formulated advantageously for injection at all.

Currently, monoclonal antibodies (mAbs) are the most popular modality of modern therapeutic proteins on the market and under development. Antibodies and antibody-like therapeutics are inherently difficult to concentrate, likely due in part to the nature of their complementarity determining regions (CDRs). Differences in CDRs among antibodies are thought to result in differences in transient protein-protein interaction propensity that manifest as bulk solution viscosity. Several groups have described the presence of reversible clusters of antibodies in viscous antibody solutions (predominantly dimers). Several theoretical descriptions of polymer viscosity have been proposed to explain the interactions of these clusters as a mechanism for bulk solution viscosity behavior.

Antibodies usually work as antagonists and, therefore, large amounts, often delivered at concentrations exceeding 100 mg/ml, are required to block undesirable interactions. For patient comfort, a single subcutaneous injection of a 1 mL volume is the most preferred mode of administration. The need to administer large amounts of antibody in a relatively small volume has required high concentration formulations at or exceeding 100 mg/ml. Antibodies are large biopolymers with molecular weights of about 150 kDa, and their high concentrations result in high sheer stress and high viscosity due to protein-protein and protein-wall interactions during filtration and passage through injection needles and in subcutaneous space. High viscosity presents challenges in the manufacture of therapeutic antigen binding proteins as well as in their administration to patients, including prohibitively high back pressure during injections leading to malfunction of injections devices, difficulty of manual administration, decreased bio-availability and patient discomfort.

The development and use of high concentration therapeutic protein solutions has accelerated as the cost of biopharmaceutical production has decreased. In some cases, these solutions possess viscous solution attributes that can make manufacturing and administration of the intended dose challenging. In therapeutic antibodies, differences in the CDRs that appear to determine if an antibody is “viscous” or “not viscous” are likely related to the propensity of the CDRs to drive protein-protein interaction and thus therapeutic effect.

Significant efforts are underway in the industry to understand the nature of interactions leading to high viscosity and to reduce the viscosity of high viscosity therapeutic protein formulations. While the invention is not limited by theory, the most important parameters affecting viscosity of the therapeutic protein formulations, particularly antibody formulations, include:
Intermolecular interactions defined by the pI of the protein and the pH of the solution. Cheng et al. (2013), “Linking the solution viscosity of an IgG2 monoclonal antibody to its structure as a function of pH and temperature,” J. Pharm Sci. 102: 4291-4304.
Charge interactions. Yadav et al. (2012), “Viscosity behavior of high-concentration monoclonal antibody solutions: correlation with interaction parameter and electroviscous effects,” J. Pharm Sci. 101: 998-1011; Yadav et al. (2012), “The influence of charge distribution on self-association and viscosity behavior of monoclonal antibody solutions.” Mol Pharm 9(4): 791-802; Singh et al. (2014), “Dipole-Dipole Interaction in Antibody Solutions: Correlation with Viscosity Behavior at High Concentration,” Pharm Res. 31(9): 2549-2558; Chaudhri et al. (2013), “The role of amino acid sequence in the self-association of therapeutic monoclonal antibodies: insights from coarse-grained modeling,” J. Phys. Chem. B 117: 1269-1279.
Hydrophobic interactions. Guo et al. (2012), “Structure-activity relationship for hydrophobic salts as viscosity-lowering excipients for concentrated solutions of monoclonal antibodies,” Pharm Res 29: 3102-3109.

The highest solution viscosity was observed under conditions with the most negative diffusion interaction parameter kD, the highest apparent radius and the lowest net charge. Neergaard et al. (2013), “Viscosity of high concentration protein formulations of monoclonal antibodies of the IgG1 and IgG4 subclass—prediction of viscosity through protein-protein interaction measurements,” Eur. J. Pharm Sci. 49: 400-410. The diffusion interaction parameter (kD), a component of the osmotic second virial coefficient (B(2)) correlated well (R>0.9) with the viscosity of concentrated mAb solutions, while the mAb net charge correlated weakly (R<0.6), indicating that weak intermolecular interactions are important in governing the viscoelastic behavior of concentrated mAb solutions. Connolly, et al. (2012), “Weak interactions govern the viscosity of concentrated antibody solutions: high-throughput analysis using the diffusion interaction parameter,” Biophys. J. 103: 69-78. Primary sequences linked to 3D structure have been used to analyze viscosity. See Honegger et al. (2001), “Yet another numbering scheme for immunoglobulin variable domains: an automatic modeling and analysis tool,” J. Mol. Biol. 309: 657-670.

Viscosity of monoclonal antibodies was assessed using molecular information in the following articles: Li, L. et al. (2014), “Concentration dependent viscosity of monoclonal antibody solutions: explaining experimental behavior in terms of molecular properties,” Pharm. Res. 31: 3161-3178; and Sharma et al. (2014), “In silico selection of therapeutic antibodies for development: viscosity, clearance, and chemical stability,” Proc. Natl. Acad. Sci. U.S.A 111: 18601-6. The net result of the interactions between antibodies is either an extended transient network of interactions (a percolating network) that result in a viscous solution or the formation of larger oligomers that then somehow influence the solution rheology as larger structures.

One strategy for reducing viscosity is to disrupt or replace viscosity-increasing protein-protein interactions with protein-excipient interactions. Excipients are additives that are included in a formulation because they either impart or enhance the stability, delivery and manufacturability of a drug product. Regardless of the reason for their inclusion, excipients are an integral component of a drug product and therefore need to be safe and well tolerated by patients. For protein drugs, the choice of excipients is particularly important because they can affect both efficacy and immunogenicity of the drug. Hence, protein formulations need to be developed with appropriate selection of excipients that afford suitable stability, safety, and marketability. It is known that single amino acids and their analogs (e.g. arginine, proline and N-acetyl arginine), some organic/inorganic salts (e.g. sodium or calcium chloride) and hydrophobic solvents, can be used as excipients to reduce the viscosity of protein solutions. However, some of these excipients do not reduce the viscosity to a desired level for ease of injection and in some cases have adverse effects on proteins, leading to destabilization and aggregate formation.

A need exists, therefore, to identify compounds that are useful for reducing viscosity of such pharmaceutical formulations, to develop methods of reducing the viscosity of such formulations, and to provide formulations with reduced viscosity. The present invention provides such methods and formulations.

### SUMMARY OF THE INVENTION

Provided in accordance with the present invention is a liquid pharmaceutical formulation comprising an antibody, an oligopeptide and a pharmaceutically acceptable buffer selected from acetate (which is preferred), glutamate, or phosphate at a pH of about 4.5 to about 6.5. The oligopeptide comprises an arginine (Arg) residue and consists of 2 to 10 amino acid residues. In preferred embodiments, the oligopeptide comprises an arginine residue at its N- or C-terminus. Further aspects of the invention disclose such a pharmaceutical formulation wherein the antibody is present in a concentration at least about 70, 85, 100, 130, 160, or 200 mg/mL, or about 200 to about 400 mg/mL. Further provided herein are preferred antibodies for such formulations.

Provided in accordance with the present invention are oligopeptides for use in the above-noted formulations at a concentration of about 10 mM to about 500 mM, with about 100 mM to about 200 mM preferred. Aspects of the invention disclose that the oligopeptide preferably is a dipeptide comprising arginine and a basic, acidic, hydrophobic, hydrophilic or aromatic residue. Residues appearing in such oligopeptides may be any of the twenty residues naturally appearing in human proteins, other naturally occurring amino acids (e.g., norleucine) or unnatural/engineered residues (e.g., D-forms of the foregoing). Aspects of the invention further disclose preferred oligopeptides as appear in Table 1 hereinafter.

Further in accordance with the present invention, other excipients may be comprised in the formulations and methods of the present invention. Although the methods and formulations of this invention may include any number of excipients known in the art, preferred embodiments comprise a surfactant, preferably polysorbate 20 or polysorbate 80. In a further aspect of the present invention, the above-noted formulations may comprise a second oligopeptide comprising arginine in a different sequence from the first oligopeptide. In a further still aspect of the present invention, the formulation may comprise an amino acid, preferably arginine or proline, n-acetyl arginine, n-acetyl lysine, n-acetyl histidine, n-acetyl proline or mixtures of any thereof.

Also provided in accordance with the present invention are methods of reducing viscosity in a pharmaceutical formulation comprising an antibody, wherein the method comprises providing a solution at a pH of about 4.5 to about 6.5 comprising (i) the antibody, (ii) an oligopeptide salt, wherein the oligopeptide comprises an arginine residue, consists of 2 to 10 amino acid residues and is present in a viscosity-reducing concentration, and (iii) a buffer. In such method, the antibody may be present in a concentration of at least about 70 mg/mL, at least about 85 mg/mL, at least about 100 mg/mL, at least about 130 mg/mL, at least about 160 mg/mL, at least about 200 mg/mL or about 200 mg/mL to about 400 mg/mL. The antibody in such method is preferably adalimumab, bevacizumab, blinatumomab, cetuximab, conatumumab, denosumab, eculizumab, erenumab, evolocumab, infliximab, natalizumab, panitumumab, rilotumumab, rituximab, romosozumab, tezepelumab, and trastuzumab, or is selected from Table 2. The oligopeptide in such method preferably has a concentration of about 100 mM to about 200 mM and the oligopeptide salt is preferably an acetate salt, which is most preferred, a sulfate salt, hydrochloride salt, or a glutamate salt. The preferred oligopeptide is selected from Arg-Arg, Arg-Lys, Arg-Phe, Arg-Pro, Arg-Val, Arg-Ala, Asp-Arg, Lys-Arg, Pro-Arg, Leu-Arg, Val-Arg, Phe-Arg, Arg-Tyr, Ala-Arg, and Arg-Arg-Arg-Arg with Pro-Arg, Phe-Arg, Arg-Arg, Arg-Phe, Arg-Val, Val-Arg, Lys-Arg, and Arg-Arg-Arg-Arg most preferred. The preferred buffers in such method are acetate and glutamate, with a preferred concentration of about 10 mM to about 50 mM.

The invention further relates to such methods and formulations wherein the solution or formulation further comprises one or more of the following:
A surfactant, preferably a polysorbate or a poloxamer with polysorbate 20 and polysorbate 80 most preferred.
A second oligopeptide comprising arginine and consisting of two to 10 amino acid residues. When the oligopeptide is a dipeptide, it may comprise along with the arginine residue a basic, acidic, hydrophobic, or aromatic residue. Preferably, the second oligopeptide is selected from Arg-Arg, Arg-Lys, Arg-Phe, Arg-Pro, Arg-Val, Arg-Ala, Asp-Arg, Lys-Arg, Pro-Arg, Leu-Arg, Val-Arg, Ala-Arg, Phe-Arg, Arg-Tyr, and Arg-Arg-Arg-Arg.
One or more amino acids, preferably selected from arginine, proline, n-acetyl arginine, n-acetyl lysine, n-acetyl histidine, n-acetyl proline or mixtures of any thereof.
A stabilizing agent as described hereinbelow.
A tonicifying agent as described hereinbelow.
An anti-oxidant as described hereinbelow.

Also provided is a method of preparing a lyophilized powder comprising the step of lyophilizing a pharmaceutical formulation as described above.

A further aspect of the invention provided herein is a lyophilized powder comprising a therapeutic protein and an oligopeptide wherein the oligopeptide comprises arginine and consists of 2 to 10 amino acids and wherein the oligopeptide is present at a weight:weight concentration effective to reduce viscosity upon reconstitution with a diluent. In one embodiment the oligopeptide is present at a concentration of about 10 μg per 1 mg of antibody, about 10 μg to about 50 μg per 1 mg of antibody, about 50 μg per mg of antibody to about 1 mg per 1 mg of antibody, about 150 μg to about 250 μg per 1 mg of antibody, and about 200 μg to about 500 μg per 1 mg of antibody. Also provided is a method for reconstituting a lyophilized powder as described above comprising the step of adding a sterile aqueous diluent comprising acetate or glutamate buffer in sufficient concentration so that the reconstituted solution has a pH of about 4 to about 8, preferably about 4.5 to about 6.

In preferred embodiments, the oligopeptide comprises an arginine residue at its N- or C-terminus. Aspects of the invention further provide that the oligopeptide used in the above-described method is present in the formulation in a concentration of about 10 mM to about 500 mM, with about 100 mM to about 200 mM preferred. Aspects of the invention disclose that the oligopeptide used in the method preferably is a dipeptide comprising a basic, acidic, hydrophobic, or aromatic residue. In further preferred embodiments of the method, the oligopeptide is selected from Table 1 hereinafter. Aspects of the invention further disclose that the oligopeptide is added to the solution as a salt, preferably an acetate salt, and is a lyophilized powder prior to being placed in solution. In one embodiment, viscosity of the formulation is reduced by at least about 30%. In another embodiment, viscosity of the formulation is reduced by at least about 50%. In a further embodiment, viscosity of the formulation is reduced by at least about 70%.

Further provided are excipients in addition to the aforementioned oligopeptides that are used in the method of the present invention. In preferred embodiments, the buffer is acetate, preferably in a concentration of about 10 to about 50 mM. Preferred embodiments further include adjusting the pH to about 5 to about 6. Aspects of the invention further disclose providing a surfactant in the formulation solution, preferably polysorbate 20 or polysorbate 80 with 0.01% w/v polysorbate concentration most preferred. Aspects of the present invention further comprise adding to the solution a second oligopeptide of 2 to 10 amino acids comprising arginine in a different sequence from the first oligopeptide. Another aspect of the present invention discloses adding an amino acid, preferably arginine or proline, n-acetyl arginine, n-acetyl lysine, n-acetyl histidine, n-acetyl proline or mixtures of any thereof.

Aspects of the invention disclose excipients designed to effectively reduce the viscosity of protein formulations that can be used to develop concentrated, low-viscosity and low volume liquid protein drug product formulations for ease of injection. The viscosity-reducing excipients identified herein are oligopeptides of 2 to 10 amino acids comprising at least one arginine. In dipeptides, the arginine residue is linked with another amino acid residue, such as basic or acidic or hydrophobic or hydrophobic/aromatic amino acid. The dipeptides and other oligopeptides may also be in reverse sequence, more specifically in the case of a dipeptide the arginine may be either at the amino or the carboxyl end of the peptide. These amino acids could interfere with viscosity-increasing protein-protein interactions through multiple types of interactions including ionic, cation-r, hydrogen bonding and hydrophobic interactions, leading to effectively reduced solution viscosity.

Aspects of this invention further contemplate reductions in viscosity in the aforementioned methods of at least about 30% and at least about 50%.

Also contemplated are methods of screening one or more formulations, each containing different concentrations of the excipient described herein to identify suitable or optimal concentrations that reduce viscosity. A method is provided for screening for a viscosity-reducing concentration of an oligopeptide comprising the steps of: (1) assessing the viscosity of a first solution comprising a first concentration of an oligopeptide of 2 to 10 amino acid residues, wherein the oligopeptide comprises arginine, and an antibody, (2) assessing the viscosity of a second solution comprising a different second concentration of the excipient and the antibody, and (3) determining that the first concentration of the oligopeptide is more viscosity-reducing than the second concentration of the oligopeptide if the first solution is less viscous. Viscosity can be determined, e.g., using a rotational viscometer such as a Gemini 200 Rheometer (Malvern Instruments) or an AR-G2 Rheometer (TA Instruments).

The invention also provides a kit comprising a liquid protein formulation of the invention, and instructions for its administration, optionally with a container, syringe and/or other administration device. Exemplary containers include vials, tubes, bottles, single or multi-chambered pre-filled syringes, or cartridges. Exemplary administration devices include syringes, with or without needles, infusion pumps, jet injectors, pen devices, transdermal injectors, or other needle-free injector.

The invention further provides a kit comprising a lyophilized powder in accordance with this invention, optionally in a container, and a sterile aqueous diluent, wherein the diluent comprises an acetate or glutamate buffer in a concentration sufficient to provide a pH of about 4 to about 8, preferably about 4.5 to about 6, in the reconstituted solution. In preferred embodiments, such a kit comprising a lyophilized powder also comprises instructions for reconstitution and administration of the antibody and a syringe or other administration device. Exemplary containers for use in the kit comprise vials, tubes, bottles, single- or multi-chambered pre-filled syringes, or cartridges. Exemplary administration devices include syringes, with or without needles, infusion pumps, jet injectors, pen devices, transdermal injectors, or other needle-free injectors, or an aerosolization device for nasal or pulmonary delivery.

### BRIEF DESCRIPTION OF THE DRAWINGS

FIG. 1 shows viscosity reduction (%) of the mAb formulation containing Arg-Phe peptide as a function of protein and peptide concentrations.

FIG. 2 shows the stability of formulations of denosumab at 120 mg/mL after 1 month at 37° C. As can be seen in the figure, both the arginine-arginine (Arg-Arg) and arginine-phenylalanine (Arg-Phe) dipeptide containing formulations reduced high molecular weight species (HMWS) formation compared to the control formulation lacking Arginine or Arg-containing dipeptides (Acetate/5% Sorbitol/PS20/pH 5.1). The Arg-Phe dipeptide formulation (10 mM Acetate, 3.6% (w/v) Sorbitol, 0.01% (w/v) Polysorbate 20, 38 mM Arginine-Phenylalanine, pH 5.2) contained the least amount of HMWS, superior to the formulation having Arg-Arg dipeptide (10 mM Acetate, 3.6% (w/v) Sorbitol, 0.01% (w/v) Polysorbate 20, 38 mM Arginine-Arginine, pH 5.1). The abbreviations in this figure refer to the formulation compositions shown in Table 9 in Example 8.

### DETAILED DESCRIPTION OF THE INVENTION

### Definition of Terms

In the description that follows, a number of terms are used extensively. The following definitions are provided to facilitate understanding of the invention.

Unless otherwise specified, “a”, “an”, “the”, and “at least one” are used interchangeably and mean one or more than one. In addition, unless otherwise required by context, singular terms shall include pluralities and plural terms shall include the singular.

As used herein, a “pharmaceutical formulation” or a “formulation” is a sterile composition of (i) a pharmaceutically active drug, such as a biologically active protein, that is suitable for parenteral administration (including but not limited to intravenous, intramuscular, subcutaneous, aerosolized, intrapulmonary, intranasal and intrathecal administration) to a patient in need thereof and (ii) one or more pharmaceutically acceptable excipients, diluents, and other additives deemed safe by the Federal Drug Administration or other foreign national authorities. Pharmaceutical formulations include liquid (e.g., aqueous) solutions that may be directly administered, and lyophilized powders that may be reconstituted into solutions by adding a diluent before administration. The term “pharmaceutical formulation” specifically excludes, however, compositions for topical administration to patients, compositions for oral ingestion, and compositions for parenteral feeding.

“Shelf life”, as used herein, means that the storage period during which an active ingredient (e.g., an antibody) in a pharmaceutical formulation has minimal degradation (e.g., not more than about 5% to 10% degradation) when the pharmaceutical formulation is stored under specified storage conditions (e.g., 2-8° C.). Techniques for assessing degradation vary depending on the identity of the protein in the pharmaceutical formulation. Exemplary techniques include size-exclusion chromatography (SEC)-HPLC to detect, for example, aggregation; reverse phase (RP)-HPLC to detect, for example, protein fragmentation; ion exchange-HPLC to detect, for example, changes in the charge of the protein; and mass spectrometry, fluorescence spectroscopy, circular dichroism (CD) spectroscopy, Fourier transform infrared spectroscopy (FT-IR), and Raman spectroscopy to detect protein conformational changes. All of these techniques can be used singly or in combination to assess the degradation of the protein in the pharmaceutical formulation and determine the shelf life of that formulation. The pharmaceutical formulations of the present invention preferably exhibit not more than about 5 to 10% increases in degradation (e.g., fragmentation, aggregation or unfolding) over two years when stored at 2-8° C.

As used herein, “viscosity” is a fluid's resistance to flow, and may be measured in units of centipoise (cP) or milliPascal-second (mPa-s), where 1 cP=1 mPa-s, at a given shear rate. Viscosity may be measured by using a rotational viscometer such as a Gemini 200 Rheometer (Malvern Instruments) or an AR-G2 Rheometer (TA Instruments). Viscosity may be measured using any other methods and in any other units known in the art (e.g., absolute, kinematic or dynamic viscosity), with the understanding that the percent reduction in viscosity afforded by use of the excipients described by the invention is what is important. Regardless of the method used to determine viscosity, the percent reduction in viscosity in excipient formulations versus control formulations will remain approximately the same at a given shear rate.

As used herein, a formulation containing an amount of an excipient effective to “reduce viscosity” (or a “viscosity-reducing” amount or concentration of such excipient) means that the viscosity of the formulation in its final form for administration is at least 5% less than the viscosity of an appropriate control formulation, such as water, buffer, other known viscosity-reducing agents such as salt and the like. Excipient-free control formulations might also be used even if they may not be implementable as a therapeutic formulation, for example due to hypotonicity.

Likewise, a “reduced viscosity” formulation is a formulation that exhibits lower viscosity compared to a control formulation.

As used herein, “stable” formulations of biologically active proteins are formulations that exhibit either (i) reduced aggregation and/or reduced loss of biological activity of at least 20% upon storage at 2-8° C. for at least 2 years compared with a control formula sample, or (ii) reduced aggregation and/or reduced loss of biological activity under conditions of thermal stress (e.g. 25° C. for 1 week to 12 weeks; 40° C. for 1 to 12 weeks; 52° C. for 7-8 days, etc.). In an embodiment, a formulation is considered stable when the protein in the formulation retains its physical stability, chemical stability and/or biological activity.

A protein may be said to “retain its physical stability” in a formulation if, for example, it shows no signs of aggregation, precipitation and/or denaturation upon visual examination of color and/or clarity, or as measured by UV light scattering or by size exclusion chromatography (SEC) or electrophoresis, such as with reference to turbidity or aggregate formation.

A protein may be said to “retain its chemical stability” in a formulation if, for example, the chemical stability at a given time is such that no new chemical entity results from modification of the protein by bond formation or cleavage. In a further embodiment, chemical stability can be assessed by detecting and quantifying chemically altered forms of the protein. Chemical alteration may involve, for example, size modification (e.g., clipping), which can be evaluated using size exclusion chromatography, SDS-PAGE and/or matrix-assisted laser desorption ionization/time-of-flight mass spectrometry (MALDI/TOF MS). Other types of chemical alteration include, for example, charge alteration (e.g., resulting from deamidation), which can be evaluated by ion-exchange chromatography. Oxidation is another commonly seen chemical modification.

A protein may be said to “retain its biological activity” in a pharmaceutical formulation relative to unmodified protein if, for example, the percentage of biological activity of the formulated protein (e.g., an antibody) as determined by an assay (e.g., an antigen binding assay) compared to the control solution is between either about 50% and about 200%, about 60% and about 170%, about 70% and about 150%, about 80% and about 125%, or about 90% and about 110%. In a further embodiment, a protein may be said to “retain its biological activity” in a pharmaceutical formulation, if, for example, without limitation, the biological activity of the protein at a given time is at least 1%, 5%, 10%, 15%, 20%, 25%, 30%, 35%, 40%, 45%, 50%, 55%, 60%, 65%, 70%, 75%, 80%, 85%, 90%, 95% or 100%.

As used herein, the terms “comprising” and “comprises” are intended to mean that the formulations and methods include the listed elements but do not exclude other unlisted elements. The terms “consisting essentially of and “consists essentially of,” when used to define formulations and methods include the listed elements, exclude unlisted elements that alter the basic nature of the formulation and/or method, but do not exclude other unlisted elements. So a formulation consisting essentially of elements defined herein would not exclude trace amounts of other elements, such as contaminants from any isolation and purification methods or pharmaceutically acceptable carriers (e.g., phosphate buffered saline), preservatives, and the like, but would exclude, for example, additional unspecified amino acids. The terms “consisting of and “consists of” when used to define formulations and methods exclude more than trace elements of other ingredients and substantial method steps for administering the compositions described herein. Embodiments defined by each of these transition terms are within the scope of this disclosure and the inventions embodied herein.

The invention concerns pharmaceutical formulations of antibodies. “Antibodies” (Abs) and the synonym “immunoglobulins” (Igs) are glycoproteins having the same structural characteristics. While antibodies exhibit binding specificity to a specific antigen, immunoglobulins include both antibodies and other antibody-like molecules that lack antigen specificity. Polypeptides of the latter kind are, for example, produced at low levels by the lymph system and at increased levels by myelomas. Thus, as used herein, the term “antibody” or “antibody peptide(s)” refers to an intact antibody, an antibody derivative, an antibody analog, a genetically altered antibody, an antibody having a detectable label, an antibody that competes for specific binding with an antibody disclosed in this specification, or an antigen-binding fragment (e.g., Fab, Fab′, F(ab′)₂, Fv, single domain antibody) thereof that competes with the intact antibody for specific binding and includes chimeric, humanized, fully human, and bispecific antibodies. In certain embodiments, antigen-binding fragments are produced, for example, by recombinant DNA techniques. In additional embodiments, antigen-binding fragments are produced by enzymatic or chemical cleavage of intact antibodies. Antigen-binding fragments include, but are not limited to, Fab, Fab′, F(ab)², F(ab′)², Fv, and single-chain antibodies.

The term “intact antibodies” as used herein refers to antibodies comprising two heavy chains and two light chains. This term thus includes without limitation fully human antibodies, genetically altered antibodies, bispecific antibodies, and antibody derivatives provided such antibodies comprised two heavy chains and two light chains.

The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

The term “isolated” as used herein refers to a protein (e.g., an antibody) that has been identified and separated and/or recovered from a component of its natural environment. Contaminant components of its natural environment are materials which would interfere with diagnostic or therapeutic uses for the protein, and may include enzymes, hormones, and other proteinaceous or nonproteinaceous solutes. In preferred embodiments, the protein will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated protein includes the protein in situ within recombinant cells since at least one component of the protein's natural environment will not be present. Ordinarily, however, isolated protein will be prepared by at least one purification step.

The monoclonal antibodies and antibody constructs formulated in accordance with the present invention specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al. (1984), Proc. Natl. Acad. Sci. USA, 8: 6851-6855). Chimeric antibodies of interest herein include “primitized” antibodies comprising variable domain antigen-binding sequences derived from a non-human primate (e.g., Old World Monkey, Ape, etc.) and human constant region sequences. A variety of approaches for making chimeric antibodies have been described. See e.g., Morrison et al. (1985), Proc. Natl. Acad. Sci. U.S.A. 81:6851; Takeda et al. (1985), Nature 314:452, Cabilly et al., U.S. Pat. No. 4,816,567; Boss et al., U.S. Pat. No. 4,816,397; Tanaguchi et al., EP 0171496; EP 0173494; and GB 2177096.

The monoclonal antibodies and antibody constructs formulated in accordance with the present invention specifically include antibodies referred to as “human” or “fully human.” The terms “human antibody” and “fully human antibody” each refer to an antibody that has an amino acid sequence of a human immunoglobulin, including antibodies isolated from human immunoglobulin libraries or from animals transgenic for one or more human immunoglobulins and that do not express endogenous immunoglobulins; for example, Xenomouse® antibodies and antibodies as described by Kucherlapati et al. in U.S. Pat. No. 5,939,598.

The term “genetically altered antibodies” means antibodies wherein the amino acid sequence has been varied from that of a native antibody. Because of the relevance of recombinant DNA techniques in the generation of antibodies, one need not be confined to the sequences of amino acids found in natural antibodies; antibodies can be redesigned to obtain desired characteristics. The possible variations are many and range from changes to just one or a few amino acids to complete redesign of, for example, the variable and/or constant region. Changes in the constant region will, in general, be made in order to improve or alter characteristics, such as complement fixation, interaction with membranes and other effector functions, as well as manufacturability and viscosity. Changes in the variable region will be made in order to improve the antigen binding characteristics.

A “Fab fragment” is comprised of one light chain and the C_{H1} and variable regions of one heavy chain. The heavy chain of a Fab molecule cannot form a disulfide bond with another heavy chain molecule.

A “Fab′ fragment” contains one light chain and one heavy chain that contains more of the constant region, between the C_{H1} and C_{H2} domains, such that an interchain disulfide bond can be formed between two heavy chains to form a F(ab′)₂ molecule.

A “F(ab′)₂ fragment” contains two light chains and two heavy chains containing a portion of the constant region between the C_{H1} and C_{H2} domains, such that an interchain disulfide bond is formed between two heavy chains.

The terms “Fv fragment” and “single chain antibody” refer to polypeptides containing antibody variable regions from both heavy and light chains but lacking constant regions. Like a whole antibody, it is able to bind selectively to a specific antigen. With a molecular weight of only about 25 kDa, Fv fragments are much smaller than common antibodies (150-160 kD) which are composed of two heavy protein chains and two light chains, and even smaller than Fab fragments (about 50 kDa, one light chain and half a heavy chain).

A “single domain antibody” is an antibody fragment consisting of a single domain Fv unit, e.g., V_{H} or V_{L}. Like a whole antibody, it is able to bind selectively to a specific antigen. With a molecular weight of only 12-15 kDa, single-domain antibodies are much smaller than common antibodies (150-160 kDa) which are composed of two heavy protein chains and two light chains, and even smaller than Fab fragments (.about.50 kDa, one light chain and half a heavy chain) and single-chain variable fragments (.about.25 kDa, two variable domains, one from a light and one from a heavy chain). The first single-domain antibodies were engineered from heavy-chain antibodies found in camelids. Although most research into single-domain antibodies is currently based on heavy chain variable domains, light chain variable domains and nanobodies derived from light chains have also been shown to bind specifically to target epitopes.

The terms “amino-terminal” and “carboxyl-terminal” and their shortened forms “N-terminus” and “C-terminus” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

One aspect of this invention concerns PCSK9 binding polypeptides. “PCSK9-binding polypeptide” means a polypeptide that binds proprotein convertase subtilisin/kexin type 9 (PCSK9) protein. In some cases, the PCSK9-binding polypeptide blocks binding of PCSK9 to low-density lipid receptors (LDLRs). Such blocking PCSK9-binding polypeptides can be monoclonal antibodies (mAbs) and can be one of the following:
a. a mAb comprising a heavy chain polypeptide having an amino acid sequence of SEQ ID NO: 1 and a light chain polypeptide having an amino acid sequence of SEQ ID NO: 2 (evolocumab), or an antigen-binding fragment thereof;
b. a mAb that competes with evolocumab for binding to PCSK9;
c. a mAb comprising:
   i. a heavy chain polypeptide comprising the following complementarity determining regions (CDRs): a heavy chain CDR1 that is a CDR1 in SEQ ID NOs: 14 or 16; a heavy chain CDR2 that is a CDR2 in SEQ ID Nos: 14 or 16; a heavy chain CDR3 that is a CDR3 in SEQ ID NOs:14 or 16, and
   ii. a light chain polypeptide comprising the following CDRs: a light chain CDR1 that is a CDR1 in SEQ ID NOs: 15 or 17; a light chain CDR2 that a CDR2 in SEQ ID NOs: 15 or 17; and a light chain CDR3 that is a CDR3 in SEQ ID NOs: 15 or 17;
d. a mAb that binds to at least one of the following residues of PCSK9, the PCSK9 comprising an amino acid sequence of SEQ ID NO: 3: S153, D188, 1189, Q190, S191, D192, R194, E197, G198, R199, V200, D224, R237, and D238, K243, S373, D374, S376, T377, F379, 1154, T1897, H193, E195, 1196, M201, V202, C223, T228, S235, G236, A239, G244, M247, 1369, S372, C375, C378, R237, and D238;
e. a mAb that binds to PCSK9 at an epitope on PCSK9 that overlaps with an epitope that is bound by an antibody that comprises:
   i. a heavy chain variable region of the amino acid sequence in SEQ ID NO: 1; and
   ii. a light chain variable region of the amino acid sequence in SEQ ID NO: 2, and
   iii. wherein the epitope of the mAb further overlaps with a site to which an epidermal growth factor-like repeat A (EGF-A) domain of the low density lipoprotein receptor (LDLR) protein (Horton, Cohen, & Hobbs (2007), Trends Biochem Sci, 32(2), 71-77. doi: 10.1016/j.tibs.2006.12.008; Seidah & Prat (2007), J Mol Med (Berl), 85(7), 685-696;
f. a mAb that comprises a heavy chain polypeptide comprising the following complementarity determining regions (CDRs):
   i. heavy chain CDR1, CDR2, and CDR3 having an amino acid sequence of SEQ ID NOs: 7, 8, and 9, respectively; and
   ii. light chain CDR1, CDR2, and CDR3 having an amino acid sequence of SEQ ID NOs: 4, 5, and 6, respectively; or
g. a mAb that comprises the heavy chain variant region sequence of SEQ ID NO: 16 and the light chain variant region sequence of SEQ ID NO: 17.

The methods and formulations of this invention concern viscosity-reducing concentrations of oligopeptides. The term “oligopeptide” as used herein refers to molecular chains of 2 to 10 amino acids joined by aminocarbonyl bonds.

As used herein, the term “amino acid” refers to either natural and/or unnatural or synthetic amino acids, including glycine and both the D and L optical isomers, amino acid analogs and peptidomimetics, including without limitation N-acetyl analogs of D or L optical isomers (e.g., N-acetyl arginine). In some aspects, the term amino acid refers to monomeric amino acids.

The terms “acidic residue” and “charged negative residue” refer to amino acid residues having sidechains comprising acidic groups. Exemplary acidic or charged negative residues include D and E.

The term “amide residue” refers to amino acids having sidechains comprising amide derivatives of acidic groups. Exemplary amide residues include N and Q.

The term “aromatic residue” refers to amino acid residues having sidechains comprising aromatic groups. Exemplary aromatic residues include F, Y, and W.

The terms “basic residue” and “charged positive residue” refer to amino acid residues having sidechains comprising basic groups. Exemplary basic or charged positive residues include H, K, and R.

The terms “hydrophilic residue” and “polar uncharged residue” refer to amino acid residues having sidechains comprising polar groups. Exemplary hydrophilic or polar uncharged residues include C, S, T, N, and Q.

The terms “non-functional residue” and “small hydrophobic residue” refer to amino acid residues having sidechains that lack acidic, basic, or aromatic groups. Exemplary non-functional, small hydrophobic residues include M, G, A, V, I, L and norleucine (Nle).

Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, for example, Sambrook et al. (2001), Molecular Cloning: A Laboratory Manual, 3rd ed., Cold Spring Harbor Laboratory Press, Cold Spring Harbor, N.Y. and Ausubel et al. (1992), Current Protocols in Molecular Biology, Greene Publishing Associates, and Harlow and Lane (1990), Antibodies: A Laboratory Manual, Cold Spring Harbor Laboratory Press, Cold Spring Harbor, N.Y. Any enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well known and commonly used in the art. Standard techniques can be used for chemical syntheses, chemical analyses, pharmaceutical preparation, formulation, and delivery, and treatment of patients.

### Amino Acids and Peptides in Pharmaceutical Formulations

Amino acids have been used in protein formulations as buffers, bulking agents, stabilizers and antioxidants. Amino acids can stabilize proteins by preferential hydration, direct binding, buffering, antioxidant properties, or other mechanisms.

Histidine is employed to buffer protein formulations in the pH range of 5.5-6.5. The imidazole group of histidine has a pKa equal to 6.0, which makes it suitable for buffering in this pH range. Histidine is commonly found in marketed protein formulations (e.g., Xolair®, Herceptin®, Recombinate®), as it is a good alternative to citrate, a buffer known to sting upon injection. Histidine is also an antioxidant that scavenges hydroxy radials in solution. Histidine has also been reported to have a stabilizing effect on ABX-IL8 (an IgG2 antibody) with respect to aggregation when used at high concentrations in both liquid and lyophilized presentations (Chen et al. (2003), Pharm Res. 20(12): 1952-60). Histidine (up to 60 mM) has also been observed to reduce the viscosity of a high concentration formulation of this antibody. In the same study, however, the authors observed increased aggregation and discoloration in histidine-containing formulations during freeze-thaw studies of the antibody in stainless steel containers. The authors attributed this effect to iron ions leached from corrosion of steel containers. Another note of caution with histidine is that it undergoes photo-oxidation in the presence of metal ions (Tomita et al. (1969), Biochemistry 8(12): 5149-60).

Glutamic acid is employed to buffer protein formulations in the pH range of 4.0-5.5. The carboxyl group of the glutamic acid side chain has a pKa of 4.3, which makes it suitable for buffering in this pH range. Acetate, the most commonly used buffer in the acidic pH range of 4.0-5.5, sublimates during lyophilization and hence should not be used in freeze-dried formulations. Glutamic acid is particularly useful in such cases (e.g., Stemgen®).

Methionine may be used as an antioxidant in pharmaceutical formulations.I It has been observed to be effective against a number of oxidative stresses (Lam et al. (1997), J Pharm Sci. 86(11): 1250-5.

Lysine stabilizes polioviruses, presumably by direct interaction with the virus.

Glycine, proline, serine and alanine have been shown to stabilize proteins by a mechanism of preferential exclusion. Glycine is also used as a buffering agent in solution. Glycine is also a commonly used bulking agent in lyophilized formulations (e.g., Neumega®, Genotropin®, Humatrope®). It crystallizes out of the frozen amorphous phase, giving the cake structure and bulk.

U.S. Pat. No. 9,364,542 B2 claims formulations comprising (i) one or more of serine, threonine, alanine, glycine and valine to stabilize the formulation together with (ii) one or more of isoleucine, asparagine, glutamine, and aspartic acid and (iii) one or more amino acids to reduce the viscosity of a formulation including proline.

Arginine has been used to inhibit aggregation in both liquid and lyophilized formulations (e.g., Activase®, Avonex®, Enbrel® liquid). The enhanced efficiency of refolding of certain proteins in the presence of arginine has been attributed to its suppression of the competing aggregation reaction during refolding. The binding of arginine by a tissue plasminogen activator (tPA) greatly increases the solubility and storage stability of an enzyme, and commercial, lyophilized formulations of PA (alteplase) contain arginine.

Patent application WO 2016/065181 discloses reducing viscosity in protein formulations using n-acetyl arginine, n-acetyl lysine, n-acetyl histidine, n-acetyl proline, and mixtures thereof at concentrations that reduce the viscosity of the protein formulations.

Patent application WO 2011/139718 alleges that certain charged amino acids and derivatives, precursors or structural analogs thereof, are useful as additives to protein-containing formulations for the purpose of reducing the viscosity of those formulations in aqueous form. In particular, the '718 application alleges that compounds capable of reducing viscosity that may be used alone or in combination with other viscosity-reducing agents are arginine (either-HCl or arginine in the presence of a succinate counterion, such as arginine succinate), arginine dipeptide, arginine tripeptide, polyarginine, homoarginine, 2-amino-3-guanidino-propionic acid, guanidine, ornithine, agmatine, guanidobutyric acid, urea, citrulline, N-hydroxy-L-nor-arginine, nitroarginine methyl ester, agininamide, arginine methyl ester, arginine ethyl ester, lysine, lysinamide, lysine methyl ester, histidine, histidine methyl ester, histamine, alanine, alaninamide, alanine methyl ester, putrescine, cadaverine, spermidine, spermine, and methionine. The disclosure further states that such compounds may be present in the formulation at a concentration that is at least 10 mM with preferences of at least 20, 50, and 100 mM. The working examples of the '718 application are limited to an anti-CD4 monoclonal antibody in 20 mM histidine-succinate buffer with various viscosity-reducing agents.

Patent application WO 2015/196091 alleges liquid formulations comprising a protein and a viscosity-reducing amount of an excipient selected from hindered amines, anionic aromatics, functionalized amino acids, oligopeptides, short-chain organic acids, and low molecular weight aliphatic polyacids. The working examples of the '091 application disclose formulations of a test protein in 50 mM histidine hydrochloride with various excipients assayed for viscosity reduction. The excipients assayed in the working examples are hindered amines, anionic aromatic compounds, guanyl taurine, oligopeptides, and various other excipients. The oligopeptides assayed in the working examples include ArgX5, RP, RK, RH, RR, and RE.

Arginine dipeptides have been used to affect insulin aggregation (Nuhu and Curtis (2015), “Arginine dipeptides affect insulin aggregation in a pH and ionic strength-dependent manner,” Biotechnol. J. 10:404-16). These authors studied the effect of certain Arg-containing dipeptides on insulin aggregation at pH 3.7, 5.5 and 7.5. They used desalted dipeptides (free-base peptides) and did not report any applications for viscosity reduction of protein formulations using these dipeptides.

### Excipients in General

The goal of protein formulation development is to transform a highly-purified drug substance (e.g., antibody) solution into a stable, efficacious biopharmaceutical dosage form for administration to patients. Kamerzell et al (2011), 63(13): 1118-59 (incorporated by reference).

One challenge in such formulations is stabilizing the product against the stresses of manufacturing, shipping and storage, which can be accomplished by certain formulation excipients. In general, excipients can be classified on the basis of the mechanisms by which they stabilize proteins against various chemical and physical stresses. Some excipients alleviate the effects of a specific stress or regulate a particular susceptibility of a specific protein. Other excipients more generally affect the physical and covalent stabilities of proteins.

Common excipients of liquid and lyophilized protein formulations appear in Table A (see Kamerzell et al. (2011), Advanced Drug Delivery Rev. 63(13): 1118-59).

TABLE A
Excipient components of protein formulations

| Excipient | | |
|---|---|---|
| component | Function | Examples |
| Buffers | Maintaining pH of solution | Citrate |
| | Buffer-ion specific interactions | Succinate |
| | with protein | Acetate |
| | | Glutamate |
| | | Aspartate |
| | | Histidine |
| | | Phosphate |
| | | Tris |
| | | Glycine |
| Sugars and | Stabilizing protein | Sucrose |
| carbohydrates | Tonicifying agents | Trehalose |
| | Carrier for inhaled drugs (lactose) | Sorbitol |
| | Dextrose solutions during IV | Mannitol |
| | administration | Glucose |
| | | Lactose |
| | | Cyclodextrin |
| | | derivatives |
| Stabilizers | Enhancing product elegance and | Mannitol |
| and bulking | preventing blowout | Glycine |
| agents | Providing structural strength to a | |
| | lyo cake | |
| Osmolytes | Stabilizing against environmental | Sucrose |
| | stress (temperature, dehydration) | Trehalose |
| | | Sorbitol |
| | | Glycine |
| | | Proline |
| | | Glutamate |
| | | Glycerol |
| | | Urea |
| Amino acids | Specific interactions with protein | Histidine |
| | Antioxidant (His, Met) | Arginine |
| | Buffering, tonicifying | Glycine |
| | | Proline |
| | | Lysine |
| | | Methionine |
| | | Aa mixtures |
| | | (e.g., glu/arg) |
| Proteins and | Competitive inhibitors of protein | HSA |
| polymers | adsorption | PVA |
| | Bulking agents for lyophilization | PVP |
| | Drug delivery vehicles | PLGA |
| | | PEG |
| | | Gelatin |
| | | Dextran |
| | | Hydroxyethyl |
| | | starch |
| | | HEC |
| | | CMC |
| Anti-oxidants | Preventing oxidative protein | Reducing agents |
| | damage | Oxygen scavengers |
| | Metal ion binders (if a metal | Free radical |
| | is included as a co-factor or is | scavengers |
| | required for protease activity) | Chelating agents |
| | Free radical scavengers | (e.g., EDTA, EGTA, |
| | | DTPA) |
| | | Ethanol |
| Metal ions | Protein co-factors | Magnesium |
| | Coordination complexes | Zinc |
| | (suspensions) | |
| Specific | Stabilizers of native conformation | Metals |
| ligands | against stress-induced unfolding | Ligands |
| | Providing conformation flexibility | Amino acids |
| | | Polyanions |
| Surfactants | Competitive inhibitor of protein | Polysorbate 20 |
| | adsorption | Polysorbate 80 |
| | Competitive inhibitor of protein | Poloxamer 188 |
| | surface denaturation | Anionic surfactants |
| | Liposomes as drug delivery | (e.g., sulfonates and |
| | vehicles | sulfosuccinates) |
| | Inhibitor of aggregation during | Cationic surfactants |
| | lyophilization | Zwitterionic |
| | Reducer of reconstitution times of | surfactants |
| | lyophilized products | |
| Salts | tonicifying agents | NaCl |
| | stabilizing or destabilizing agents | KCl |
| | for proteins, especially anions | NaSO₄ |
| Preservatives | Protection against microbial | Benzyl alcohol |
| | growth in formulation | M-cresol |
| | | Phenol |

Other excipients are known in the art and can be found in Powell et al. (1998), “Compendium of Excipients for Parenteral Formulations,” PDA J. Pharm. Sci. Tech., 52:238-311, which is hereby incorporated by reference.

Given the teachings and guidance provided herein, those skilled in the art will know what amount or range of excipient can be included in any particular formulation to achieve a biopharmaceutical formulation of the invention. For example, the amount and type of a salt to be included in a biopharmaceutical formulation of the invention can be selected based on the desired osmolality (i.e., isotonic, hypotonic or hypertonic) of the final solution as well as the amounts and osmolality of other components to be included in the formulation. Similarly, by exemplification with reference to the type of polyol or sugar included in a formulation, the amount of such an excipient will depend on its osmolality.

Those skilled in the art can determine what amount or range of excipient can be included in any particular formulation to achieve a biopharmaceutical formulation of the invention that promotes retention in stability of the biopharmaceutical. For example, the amount and type of a salt to be included in a biopharmaceutical formulation of the invention can be selected based on to the desired osmolality (i.e., isotonic, hypotonic or hypertonic) of the final solution as well as the amounts and osmolality of other components to be included in the formulation. Similarly, by exemplification with reference to the type of polyol or sugar included in a formulation, the amount of such an excipient will depend on its osmolality.

About 5% (weight/volume) sorbitol, for example, can achieve isotonicity while about 9% (weight/volume) of a sucrose excipient is needed to achieve isotonicity. Selection of the amount or range of concentrations of one or more excipients that can be included within a biopharmaceutical formulation of the invention has been exemplified above by reference to salts, polyols and sugars. However, those skilled in the art will understand that the considerations described herein and further exemplified by reference to specific excipients are equally applicable to all types and combinations of excipients including, for example, salts, amino acids, other tonicity agents, surfactants, stabilizers, bulking agents, cryoprotectants, lyoprotectants, anti-oxidants, metal ions, chelating agents and/or preservatives.

Further, where a particular excipient is reported in a formulation by, e.g., percent (%) w/v, those skilled in the art will recognize that the equivalent molar concentration of that excipient is also contemplated.

Those having ordinary skill in the art would recognize that the concentrations of the aforementioned excipients share an interdependency within a particular formulation. By way of example, the concentration of a bulking agent may be lowered where, for example, there is a high protein/peptide concentration or a high stabilizing agent concentration. In addition, a person having ordinary skill in the art would recognize that, in order to maintain the isotonicity of a particular formulation in which there is no bulking agent, the concentration of a stabilizing agent would be adjusted accordingly (i.e., a “tonicifying” amount of stabilizer would be used).

### Buffers

Solution pH affects the chemical integrity of a protein's amino acid residues (e.g., Asn deamidation and Met oxidation) and maintenance of its higher order structure. Those skilled in the art thus use buffering agents to control solution pH and optimize protein stability. Maximal stability of a protein drug is usually within a narrow pH range. Several approaches (e.g., accelerated stability studies and calorimetric screening studies) are useful for this purpose (Remmele et al. (1999), Biochemistry, 38(16): 5241-7). Once a formulation is finalized, the drug product must be manufactured and maintained within a predefined specification throughout its shelf-life. Hence, buffering agents are almost always employed to control pH in the formulation.

Organic acids, phosphates and Tris have been employed routinely as buffers in protein formulations (see Table B). The buffer capacity of the buffering species is maximal at a pH equal to the pKa and decreases as pH increases or decreases away from this value. Ninety percent of the buffering capacity exists within one pH unit of its pKa. Buffer capacity also increases proportionally with increasing buffer concentration.

TABLE B
Commonly used buffering agents and their pKₐ values

| Buffer | pKₐ | Example drug product |
|---|---|---|
| Acetate | 4.8 | Neupogen ®, Neulasta ® |
| Succinate | pKₐ₁ = 4.8, pKₐ₂ = 5.5 | Actimmune ® |
| Citrate | pKₐ₁ = 3.1, pKₐ₂ = 4.8, | Humira ® |
| | pKₐ₃ = 6.4 | |
| Histidine | 6.0 | Xolair ® |
| (imidazole) | | |
| phosphate | pKₐ₁ = 2.15, pKₐ₂ = 7.2, | Enbrel ® (liquid formulation) |
| | pKₐ₃ = 12.3 | |
| Tris | 8.1 | Leukine |

In addition to the foregoing, some therapeutic proteins may be self-buffering at a pharmaceutically relevant concentration. Formulations of such proteins might not need to include a conventional buffer at all. See US patent application 2012/0028877, which is hereby incorporated by reference.

More typically, a pH buffering compound may be present in any amount suitable to maintain the pH of the formulation at a predetermined level. In one embodiment, when the pH buffering agent is an amino acid, the concentration of the amino acid is between 0.1 mM and 1000 mM (1 M). In one embodiment, the pH buffering agent is at least 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, 90, 100, 200, 500, 700, or 900 mM. In another embodiment, the concentration of the pH buffering agent is between 1, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, or 90 mM and 100 mM. In still another embodiment, the concentration of the pH buffering agent is between 5, 6, 7, 8, 9, 10, 11, 12, 13, 14, 15, 16, 17, 18, 19, 20, 30, or 40 mM and 50 mM. In yet another embodiment, the concentration of the pH buffering agent is 10 mM.

### Sugars and Carbohydrates

Sugars are frequently used to stabilize proteins in both liquid and lyophilized formulations. Disaccharides such as sucrose and trehalose are thought to stabilize proteins by preferential hydration at high concentrations in the liquid state and by specific interactions with proteins and formation of viscous glassy matrices in the solid state. Sugar molecules can increase the viscosity of monoclonal antibody solutions, presumably due to a preferential hydration mechanism. Sugar alcohols such as sorbitol can stabilize proteins in solution and in the lyophilized state. Mannitol is often used as a bulking agent in lyophilized formulations. Lactose is used as a carrier molecule for inhaled formulations of proteins. Cyclodextrin derivatives can stabilize proteins in liquid formulations of antibodies, vaccine antigens, and such smaller proteins as growth factors, interleukin-2 and insulin.

### Stabilizers and Bulking Agents

Bulking agents are typically used in lyophilized formulations to enhance product elegance and to prevent blowout. Conditions in the formulation are generally designed so that the bulking agent crystallizes out of the frozen amorphous phase (either during freezing or annealing above the Tg′) giving the cake structure and bulk. Mannitol and glycine are examples of commonly used bulking agents.

Stabilizers include a class of compounds that can serve as cryoprotectants, lyoprotectants, and glass forming agents. Cryoprotectants act to stabilize proteins during freezing or in the frozen state at low temperatures (P. Cameron, ed., Good Pharmaceutical Freeze-Drying Practice, Interpharm Press, Inc., Buffalo Grove, IL, (1997)). Lyoprotectants stabilize proteins in the freeze-dried solid dosage form by preserving the native-like conformational properties of the protein during dehydration stages of freeze-drying. Glassy state properties have been classified as “strong” or “fragile” depending on their relaxation properties as a function of temperature. It is important that cryoprotectants, lyoprotectants, and glass forming agents remain in the same phase with the protein in order to impart stability. Sugars, polymers, and polyols fall into this category and can sometimes serve all three roles.

Polyols encompass a class of excipients that includes sugars, (e.g. mannitol, sucrose, sorbitol), and other polyhydric alcohols (e.g., glycerol and propylene glycol). The polymer polyethylene glycol (PEG) is included in this category. Polyols are commonly used as stabilizing excipients and/or isotonicity agents in both liquid and lyophilized parenteral protein formulations. With respect to the Hofmeister series, the polyols are kosmotropic and are preferentially excluded from the protein surface. Polyols can protect proteins from both physical and chemical degradation pathways. Preferentially excluded co-solvents increase the effective surface tension of solvent at the protein interface whereby the most energetically favorable protein conformations are those with the smallest surface areas.

Mannitol is a popular bulking agent in lyophilized formulations because it crystallizes out of the amorphous protein phase during freeze-drying lending structural stability to the cake (e.g. Leukine®, Enbrel®-Lyo, Betaseron®). It is generally used in combination with a cryo and/or lyoprotectant like sucrose. Because of the propensity of mannitol to crystallize under frozen conditions, sorbitol and sucrose are the preferred tonicity agents/stabilizers in liquid formulations to protect the product against freeze-thaw stresses encountered during transport or when freezing bulk prior to manufacturing. Sorbitol and sucrose are far more resistant to crystallization and therefore less likely to phase separate from the protein. It is interesting to note that while mannitol has been used in tonicifying amounts in several marketed liquid formulations such as Actimmune®, Forteo®, and Rebif®, the product labels of these drugs carry a ‘Do Not Freeze’ warning. The use of reducing sugars (containing free aldehyde or ketone groups) such as glucose and lactose should be avoided because they can react and glycate surface lysine and arginine residues of proteins via the Maillard reaction of aldehydes and primary amines (Chevalier F, et al., Nahrung, 46(2): 58-63 (2002); Humeny A, et al., J Agric Food Chem. 50(7): 2153-60 (2002)). Sucrose can hydrolyze to fructose and glucose under acidic conditions (Kautz C. F. and Robinson A. L., JACS, 50(4) 1022-30 (1928)), and consequently may cause glycation.

In particular embodiments of the present compositions, a stabilizer (or a combination of stabilizers) is added to a lyophilization formulation to prevent or reduce lyophilization-induced or storage-induced aggregation and chemical degradation. A hazy or turbid solution upon reconstitution indicates that the protein has precipitated. The term “stabilizer” means an excipient capable of preventing aggregation or other physical degradation, as well as chemical degradation (for example, autolysis, deamidation, oxidation, etc.) in an aqueous and solid state. Stabilizers that are conventionally employed in pharmaceutical compositions include, but are not limited to, sucrose, trehalose, mannose, maltose, lactose, glucose, raffinose, cellobiose, gentiobiose, isomaltose, arabinose, glucosamine, fructose, mannitol, sorbitol, glycine, arginine HCL, poly-hydroxy compounds, including polysaccharides such as dextran, starch, hydroxyethyl starch, cyclodextrins, N-methyl pyrollidene, cellulose and hyaluronic acid, sodium chloride, Carpenter et al. (1991), Develop. Biol. Standard 74:225.

### Osmolytes

Osmolytes currently used as protein formulation excipients are listed in Table A. Other osmolytes commonly found in nature that may be useful as excipients include taurine, betaine, trimethylamine N-oxide (TMAO), choline-O-sulfate, and sarcosine.

### Proteins and Polymers

Protein-based excipients add complexity to the formulation, especially in developing analytical methods to monitor the stability of the protein-based drug or vaccine in the presence of a protein-based excipient. Polymers have been evaluated as excipients (e.g., as bulking agents) in lyophilized protein formulations. Controlled release formulations of protein drugs and vaccines are being studied in which proteins are formulated with polymers such as PLGA and PEG. Many additional water-soluble polymers (e.g., HEC, CMC have been utilized for topical formulations of protein drugs.

PEG can stabilize proteins by two different temperature-dependent mechanisms. At lower temperatures, it is preferentially excluded from the protein surface but has been shown to interact with the unfolded form of the protein at higher temperature given its amphipathic nature (Lee and Lee (1987), Biochemistry, 26(24): 7813-9). It may protect proteins via preferential exclusion at lower temperatures but possibly by reducing the number of productive collisions between unfolded molecules at higher temperatures. PEG is also a cryoprotectant and has been employed in Recombinate®, a lyophilized formulation of recombinant Antihemophilic Factor.

### Anti-Oxidants

Many different sources may oxidize protein residues. Oxidative protein damage can be minimized by carefully controlling the manufacturing process and storage of the product, including such factors as atmospheric oxygen, temperature, light exposure, and chemical contamination. Where such controls are inadequate, anti-oxidant excipients can be included in the formulation.

The most commonly used pharmaceutical antioxidant excipients are reducing agents, oxygen/free-radical scavengers, or chelating agents. Antioxidants in therapeutic protein formulations must be water-soluble and remain active throughout the product shelf-life. Reducing agents and oxygen/free-radical scavengers work by ablating active oxygen species in solution. Chelating agents (e.g., EDTA) can be effective by binding trace metal contaminants that promote free-radical formation. In the liquid formulation of acidic fibroblast growth factor, for example, EDTA inhibits metal ion-catalyzed oxidation of cysteine residues. EDTA has been used in marketed products like Kineret® and Ontak®.

One caution is that antioxidants themselves may induce other covalent or physical changes to the protein. Reducing agents (e.g., glutathione) can cause disruption of intramolecular disulfide linkages, which can lead to disulfide shuffling. In the presence of transition metal ions, ascorbic acid and EDTA have been shown to promote methionine oxidation in a number of proteins and peptides (Akers and Defelippis (1999), “Peptides and Proteins as Parenteral Solutions” in Pharmaceutical Formulation Development of Peptides and Proteins (Frokjaer and Hovgaard, eds.; Taylor and Francis (1999), Pharmaceutical Science; Fransson (1997), J. Pharm. Sci. 86(9): 4046-50; Yin et al. (2004), Pharm Res., 21(12): 2377-83). Sodium thiosulfate has been reported to reduce the levels of light- and temperature-induced methionine oxidation in rhuMab HER2; however, the formation of a thiosulfate-protein adduct was also reported in this study (Lam et al. (1997), J. Pharm Sci. 86(11): 1250-5). Selection of an appropriate antioxidant is made according to the specific stresses and sensitivities of the protein.

### Metal Ions

In general, transition metal ions are undesired in protein formulations because they can catalyze physical and chemical degradation reactions in proteins. Specific metal ions are included in formulations, however, when they act as co-factors to proteins. Metal ions may also be used in suspension formulations of proteins where they form coordination complexes (e.g., zinc suspension of insulin). The use of magnesium ions (10-120 mM) has been proposed to inhibit the isomerization of aspartic acid to isoaspartic acid (WO 2004/039337).

Metal ions were found to confer stability and/or increased activity in a formulation of human deoxyribonuclease (rhDNase, Pulmozyme®). Ca⁺² ions (up to 100 mM) increased the stability of the enzyme through a specific binding site (Chen et al. (1999), J Pharm Sci. 88(4): 477-82). In fact, removal of calcium ions from the solution with EGTA caused an increase in deamidation and aggregation. However, this effect was observed only with Ca⁺² ions; other divalent cations—Mg⁺², Mn⁺² and Zn⁺²—were observed to destabilize rhDNase.

Similar effects were observed in formulation of Factor VIII. Ca⁺² and Sr⁺² ions stabilized the protein while others like Mg⁺², Mn⁺² and Zn⁺², Cu⁺² and Fe⁺² destabilized it (Fatouros, et al. (1997), Int. J. Pharm., 155, 121-131). In a separate study with Factor VIII, a significant increase in aggregation rate was observed in the presence of Al⁺³ ions (Derrick et al. (2004), J. Pharm. Sci., 93(10): 2549-57). The authors note that other excipients like buffer salts are often contaminated with Al⁺³ ions and illustrate the need to use excipients of appropriate quality in formulated products. Vaccines containing live or killed attenuated picornaviruses, such as Hepatitis A and polio, are conformationally stabilized by magnesium. Metal ions such as calcium, magnesium and zinc improve the stability of oxytocin in an aqueous solution Insulin can bind zinc, leading to the formation of dimers and hexamers in a crystalline form, which allows for the preparation of different formulations with different in vivo release profiles. The chemical and thermal stability of the hexamer insulin formulation varies in the presence of different levels of zinc and phenol.

### Specific Ligands

One approach to improve the conformational stability of protein therapeutic drugs is to take advantage of the protein's inherent ligand binding sites. For example, Pulmozyme® not only requires bivalent metal ions for its enzymatic activity, it has improved conformational stability in the presence of calcium ions. Both acidic and basic fibroblast growth factors (aFGF and bFGF) have been evaluated clinically for their ability to promote wound healing, and both proteins naturally bind to the highly negatively charged proteoglycans on cell surfaces. A variety of other highly negatively charged compounds also bind and dramatically stabilize aFGF by interaction with the protein's polyanion binding site.

### Surfactants

Protein molecules have a high propensity to interact with surfaces, making them susceptible to adsorption and denaturation at air-liquid, vial-liquid, and liquid-liquid (silicone oil) interfaces. This degradation pathway is inversely dependent on protein concentration and results in soluble or insoluble protein aggregates or the loss of protein from solution through adsorption to surfaces. In addition to container surface adsorption, surface-induced degradation is exacerbated with physical agitation, as would be experienced during shipping and handling.

Surfactants are commonly used in protein formulations to prevent surface-induced degradation. Surfactants are amphipathic molecules with the capability of out-competing proteins for interfacial positions. Hydrophobic portions of the surfactant molecules occupy interfacial positions (e.g., air/liquid), while hydrophilic portions of the molecules remain oriented towards the bulk solvent. At sufficient concentrations (typically around the detergent's critical micellar concentration), a surface layer of surfactant molecules serve to prevent protein molecules from adsorbing at the interface. Thereby, surface-induced degradation is minimized.

The most commonly used surfactants are the non-ionic fatty acid esters of sorbitan polyethoxylates—i.e., polysorbate 20 and polysorbate 80 (e.g., in the drug products Avonex®, Neupogen®, Neulasta®). The two differ only in the length of the aliphatic chain that imparts hydrophobic character to the molecules, C-12 and C-18, respectively. Polysorbate 80 is more surface-active and has a lower critical micellar concentration than polysorbate 20. Both polysorbate 20 and polysorbate 80 have been shown to protect against agitation-induced aggregation. Polysorbate 20 and 80 also protect against stress induced by freezing, lyophilization and reconstitution. Both polysorbate 20 and 80 may contain peroxides which can oxidize proteins and they themselves may degrade by either oxidation or hydrolysis with varying effects on protein stability. It can also be difficult to control the level of polysorbate 20 or 80 in formulations due to their complex behavior during membrane filtration (especially at concentrations in which polysorbates form micelles in solution). The surfactant poloxamer 188 has also been used in several marketed liquid products, such Gonal-F®, Norditropin®, and Ovidrel®. It is generally believed that non-ionic surfactants stabilize proteins primarily by outcompeting protein molecules for hydrophobic surfaces (e.g., air-water interfaces), thereby preventing proteins from unfolding at these hydrophobic interfaces. Non-ionic surfactants can also block protein molecules from adsorbing to other hydrophobic surfaces present during processing. In addition, non-ionic surfactants may directly interact with hydrophobic regions in protein molecules. Monoclonal antibodies can affect the critical micelle concentration of polysorbate 20 compared to buffer alone.

Detergents can also affect the thermodynamic conformational stability of proteins. Here again, the effects of a given excipient will be protein-specific. For example, polysorbates have been shown to reduce the stability of some proteins and increase the stability of others. Detergent destabilization of proteins can be rationalized in terms of the hydrophobic tails of the detergent molecules that can engage in specific binding with partially or wholly unfolded protein states. These types of interactions could cause a shift in the conformational equilibrium towards the more expanded protein states (i.e., increasing the exposure of hydrophobic portions of the protein molecule in complement to binding polysorbate). Alternatively, if the protein native state exhibits some hydrophobic surfaces, detergent binding to the native state may stabilize that conformation.

Another aspect of polysorbates is that they are inherently susceptible to oxidative degradation. Often, as raw materials, they contain sufficient quantities of peroxides to cause oxidation of protein residue side-chains, especially methionine. The potential for oxidative damage arising from the addition of stabilizer emphasizes the point that the lowest effective concentrations of excipients should be used in formulations. For surfactants, the effective concentration for a given protein will depend on the mechanism of stabilization. It has been postulated that if the mechanism of surfactant stabilization is related to preventing surface denaturation, then the effective concentration will be around the detergent's critical micellar concentration. Conversely, if the mechanism of stabilization is associated with specific protein-detergent interactions, the effective surfactant concentration will be related to the protein concentration and the stoichiometry of the interaction (Randolph et al. (2002), Pharm Biotechnol., 13:159-75).

Surfactants may also be added in appropriate amounts to prevent surface-related aggregation during freezing and drying (Chang (1996), J. Pharm. Sci. 85:1325). Exemplary surfactants include anionic, cationic, nonionic, zwitterionic, and amphoteric surfactants, including surfactants derived from naturally occurring amino acids. Anionic surfactants include, but are not limited to, sodium lauryl sulfate, dioctyl sodium sulfosuccinate and dioctyl sodium sulfonate, chenodeoxycholic acid, N-lauroylsarcosine sodium salt, lithium dodecyl sulfate, 1-octanesulfonic acid sodium salt, sodium cholate hydrate, sodium deoxycholate, and glycodeoxycholic acid sodium salt. Cationic surfactants include, but are not limited to, benzalkonium chloride or benzethonium chloride, cetylpyridinium chloride monohydrate, and hexadecyltrimethylammonium bromide. Zwitterionic surfactants include, but are not limited to, CHAPS, CHAPSO, SB3-10, and SB3-12. Non-ionic surfactants include, but are not limited to, digitonin, Triton X-100, Triton X-114, TWEEN-20, and TWEEN-80. In another embodiment, surfactants include lauromacrogol 400, polyoxyl 40 stearate, polyoxyethylene hydrogenated castor oil 10, 40, 50 and 60, glycerol monostearate, polysorbate 40, 60, 65 and 80, soy lecithin and other phospholipids such as DOPC, DMPG, DMPC, and DOPG; sucrose fatty acid ester, methyl cellulose and carboxymethyl cellulose. In one embodiment, the surfactant is incorporated in a concentration of about 0% to about 5% w/v. In another embodiment, the surfactant is incorporated in a concentration of at least 0.001, 0.002, 0.004, 0.005, 0.007, 0.01, 0.05, 0.1, 0.2, 0.3, 0.4, 0.5, 0.6, 0.7, 0.7, 0.8, 0.9, 1.0, 1.5, 2.0, 2.5, 3.0, 3.5, 4.0, or 4.5% w/v. In another embodiment, the surfactant is incorporated in a concentration of about 0.001% to about 0.5% w/v. In still another embodiment, the surfactant is incorporated in a concentration of about 0.004, 0.005, 0.007, 0.01, 0.05, or 0.1% w/v to about 0.2% w/v. In yet another embodiment, the surfactant is incorporated in a concentration of about 0.01% to about 0.1% w/v.

### Salts

Salts are often added to increase the ionic strength of the formulation, which can be important for protein solubility, physical stability, and isotonicity. Salts can affect the physical stability of proteins in a variety of ways. Ions can stabilize the native state of proteins by binding to charged residues on the protein's surface. Alternatively, they can stabilize the denatured state by binding to the peptide groups along the protein backbone (—CONH—). Salts can also stabilize the protein native conformation by shielding repulsive electrostatic interactions between residues within a protein molecule. Electrolytes in protein formulations can also shield attractive electrostatic interactions between protein molecules that can lead to protein aggregation and insolubility.

The effect of salt on the stability and solubility of proteins varies significantly with the characteristics of the ionic species. The Hofmeister series originated in the 1880's as a way to rank order electrolytes based on their ability to precipitate proteins (Cacace et al. (1997), Quarterly Reviews of Biophysics, 30(3): 241-277). In this report, the Hofmeister series is used to illustrate a scale of protein stabilization effects by ionic and non-ionic co-solutes. In Table C, co-solutes are ordered with respect to their general effects on solution state proteins, from stabilizing (kosmotropic) to destabilizing (chaotropic). In general, the differences in effects across the anions are far greater than that observed for the cations, and, for both types, the effects are most apparent at higher concentrations than are acceptable in parenteral formulations. High concentrations of kosmotropes (e.g., >1 molar ammonium sulfate) are commonly used to precipitate proteins from solution by a process called ‘salting-out’ where the kosmotrope is preferentially excluded from the protein surface reducing the solubility of the protein in it's native (folded) conformation. Removal or dilution of the salt will return the protein to solution. The term ‘salting-in’ refers to the use of destabilizing ions (e.g., like guanidine and chloride) that increase the solubility of proteins by solvating the peptide bonds of the protein backbone. Increasing concentrations of the chaotrope will favor the denatured (unfolded) state conformation of the protein as the solubility of the peptide chain increases. The relative effectiveness of ions to ‘salt-in’ and ‘salt-out’ defines their position in the Hofmeister series.

TABLE C
The Hofmeister series of salts

| Cosolute | | | | |
|---|---|---|---|---|
| Anion | Cation | Other | Stabilization scales | |
| F⁻ | (CH₃)₄N⁺ | Glycerol/Sorbitol | Stabilizing | Kosmotropic |
| PO₄_{^{−}} | (CH₃)₂NH⁺ | Sucrose/Trehalose | (salting-out) | [symbol] |
| SO₄_{^{−}} | NH₄₊ | TMAO | [symbol] | |
| CHCOO⁻ | K⁺ | | | |
| Cl⁻ | Na⁺ | | | |
| Br⁻ | Cs⁺ | | | |
| I⁻ | Li⁺ | | | |
| | Mg²⁺ | Guanidine | | |
| | Ca²⁺ | Arginine | Destabilizing | Chaotropic |
| | Ba²⁺ | Urea | (salting-in) | |

In order to maintain isotonicity in a parenteral formulation, salt concentrations are generally limited to less than 150 mM for monovalent ion combinations. In this concentration range, the mechanism of salt stabilization is probably due to screening of electrostatic repulsive intramolecular forces or attractive intermolecular forces (Debye-Huckel screening). Interestingly, chaotropic salts have been shown to be more effective at stabilizing the protein structure than similar concentrations of kosmotropes by this mechanism. The chaotropic anions are believed to bind more strongly than the kosmotropic ions. With respect to covalent protein degradation, differential effects of ionic strength on this mechanism are expected through Debye-Huckel theory. Accordingly, published reports of protein stabilization by sodium chloride are accompanied by those where sodium chloride accelerated covalent degradation. The mechanisms by which salts affect protein stability are protein specific and may vary significantly as a function of solution pH. An example where an excipient can be useful in enabling the delivery of a protein drug is that of some high concentration antibody formulations. Over the last several years, salts have been shown to be effective in reducing the viscosity of such formulations (Liu et al. (2005, 2006), J. Pharm Sci., 94(9): 1928-40, erratum in J Pharm Sci., 95(1): 234-5.

### Preservatives

Preservatives are necessary when developing multi-use parenteral formulations that involve more than one extraction from the same container. Their primary function is to inhibit microbial growth and ensure product sterility throughout the shelf-life or term of use of the drug product. Commonly used preservatives include benzyl alcohol, phenol and m-cresol. Although preservatives have a long history of use, the development of protein formulations that includes preservatives can be challenging. Preservatives almost always have a destabilizing effect (aggregation) on proteins, and this has become a major factor in limiting their use in multi-dose protein formulations (Roy et al. (2005), J. Pharm. Sci., 94(2): 382-96). Benzyl alcohol has also been shown to affect protein structure and stability in a concentration-, temperature- and time-dependent manner. Due to these destabilizing effects, many lyophilized protein formulations are reconstituted with diluent containing benzyl alcohol to minimize the contact time with the protein prior to administration.

Most protein drugs have been formulated for single-use only. However, when multi-dose formulations are possible, they have the added advantage of enabling patient convenience, and increased marketability. A good example is that of human growth hormone (hGH) where the development of preserved formulations has led to commercialization of more convenient, multi-use injection pen presentations. At least four such pen devices containing preserved formulations of hGH are currently available. Norditropin® (liquid), Nutropin AQ® (liquid) & Genotropin (lyophilized—dual chamber cartridge) contain phenol while Somatrope® is formulated with m-cresol.

Several aspects need to be considered during the formulation development of preserved dosage forms. The effective preservative concentration in the drug product must be optimized. This requires testing a given preservative in the dosage form with concentration ranges that confer anti-microbial effectiveness without compromising protein stability. For example, three preservatives were successfully screened in the development of a liquid formulation for interleukin-1 receptor (Type I), using differential scanning calorimetry (DSC). The preservatives were rank-ordered based on their impact on stability at concentrations commonly used in marketed products (Remmele et al. (1998), Pharm. Res., 15(2): 200-8).

As might be expected, development of liquid formulations containing preservatives are more challenging than lyophilized formulations. Freeze-dried products can be lyophilized without the preservative and reconstituted with a preservative containing diluent at the time of use. This shortens the time during which a preservative is in contact with the protein, thus significantly minimizing the associated stability risks. With liquid formulations, preservative effectiveness and stability have to be maintained over the entire product shelf-life (usually about 18-24 months). An important point to note is that preservative effectiveness has to be demonstrated in the final formulation containing the active drug and all excipient components.

Some preservatives can cause injection site reactions, which is another factor that needs consideration when choosing a preservative. In clinical trials that focused on the evaluation of preservatives and buffers in Norditropin®, pain perception was observed to be lower in formulations containing phenol and benzyl alcohol as compared to a formulation containing m-cresol (Kappelgaard (2004), Horm. Res. 62 Suppl 3:98-103). Interestingly, among the commonly used preservative, benzyl alcohol possesses anesthetic properties (Minogue and Sun (2005), Anesth. Analg. 100(3): 683-6).

### Viscosity-Reducing Excipients of the Present Invention

Reducing the viscosity of therapeutic antibody formulations is of interest to the pharmaceutical industry. The oligopeptide excipients of this invention were discovered to reduce the viscosity of such formulations. The invention provides the excipient at selected concentrations for use in reducing the viscosity of antibody formulations. Provided herein are antibody formulations and methods for reducing the viscosity of antibody formulations by combining the antibody with a viscosity-reducing concentration of an oligopeptide of 2 to 10 amino acid residues comprising arginine.

Table 1 lists exemplary viscosity-reducing excipients in accordance with this invention.

TABLE 1
Viscosity-reducing excipients

| Excipient | | Functional | |
|---|---|---|---|
| ID | Excipients | group linked | Peptide Structure |
| 1 | H-Arg-Arg-OH (acetate salt) | Basic | 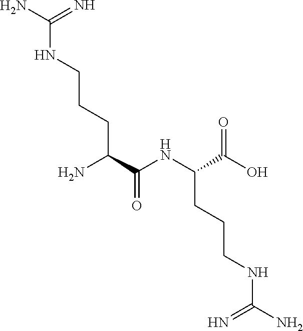 [SMILES: N=C(N)NCCC[C@H](NC(=O)[C@@H](N)CCCNC(=N)N)C(=O)O] |
| 2 | H-Arg-Lys-OH (acetate salt) | Basic (link to carboxyl end of Arg) | 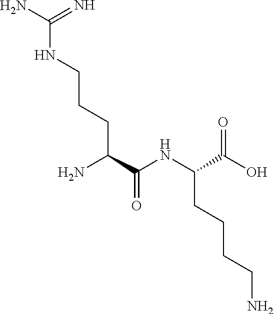 [SMILES: N=C(N)NCCC[C@H](N)C(=O)N[C@@H](CCCCN)C(=O)O] |
| 3 | H-Arg-Phe-OH (acetate salt) | Aromatic/ hydrophobic (link to carboxyl end of Arg) | 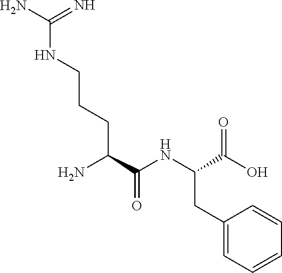 [SMILES: N=C(N)NCCC[C@H](N)C(=O)N[C@@H](Cc1ccccc1)C(=O)O] |
| 4 | H-Arg-Tyr-OH (acetate salt) | Aromatic/ hydrophobic (link to carboxyl end of Arg) | 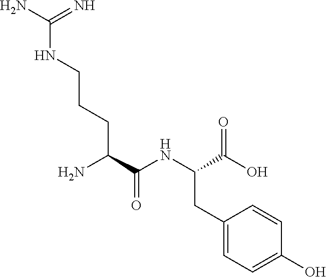 [SMILES: N=C(N)NCCC[C@H](N)C(=O)N[C@@H](Cc1ccc(O)cc1)C(=O)O] |
| 5 | H-Arg-Pro-OH (acetate salt or sulfate salt) | Hydrophobic (link to carboxyl end of Arg) | 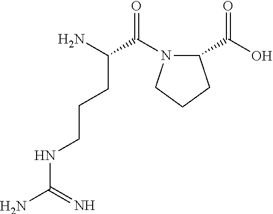 [SMILES: N=C(N)NCCC[C@H](N)C(=O)N1CCC[C@H]1C(=O)O] |
| 6 | H-Arg-Val-OH (acetate salt) | Hydrophobic (link to carboxyl end of Arg) | 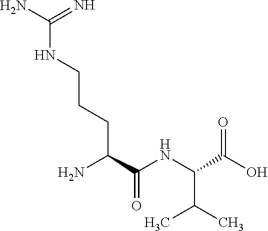 [SMILES: CC(C)[C@H](NC(=O)[C@@H](N)CCCNC(=N)N)C(=O)O] |
| 7 | H-Arg-Ala-OH (acetate salt) | Hydrophobic (link to carboxyl end of Arg) | 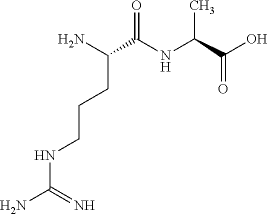 [SMILES: C[C@H](NC(=O)[C@@H](N)CCCNC(=N)N)C(=O)O] |
| 8 | H-Asp-Arg-OH (acetate salt) | Acidic (link to carboxyl end of Arg) | 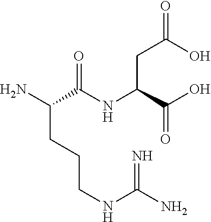 [SMILES: N=C(N)NCCC[C@H](N)C(=O)N[C@@H](CC(=O)O)C(=O)O] |
| 9 | H-Lys-Arg-OH (acetate salt) | Basic (link to amino end of Arg) | 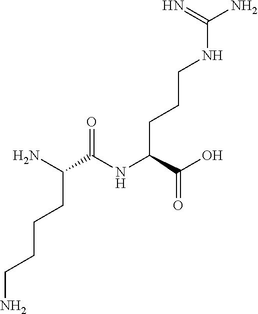 [SMILES: N=C(N)NCCC[C@H](NC(=O)[C@@H](N)CCCCN)C(=O)O] |
| 10 | H-Phe-Arg-OH (acetate salt) | Aromatic/ hydrophobic (link to amino end of Arg) | 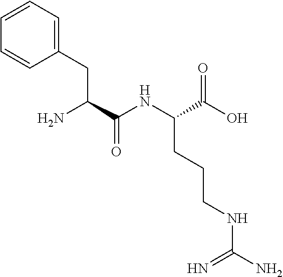 [SMILES: N=C(N)NCCC[C@H](NC(=O)[C@@H](N)Cc1ccccc1)C(=O)O] |
| 11 | H-Pro-Arg-OH (acetate salt) | Hydrophobic (link to amino end of Arg) | 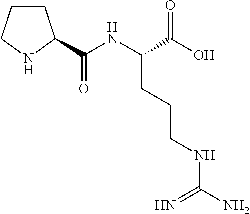 [SMILES: N=C(N)NCCC[C@H](NC(=O)[C@@H]1CCCN1)C(=O)O] |
| 12 | H-Leu-Arg-OH (acetate salt) | Hydrophobic (link to amino end of Arg) | 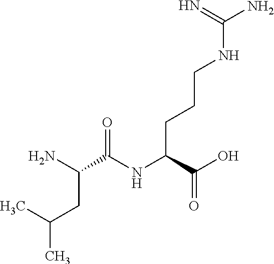 [SMILES: CC(C)C[C@H](N)C(=O)N[C@@H](CCCNC(=N)N)C(=O)O] |
| 13 | H-Val-Arg-OH (acetate salt) | Hydrophobic (link to amino end of Arg) | 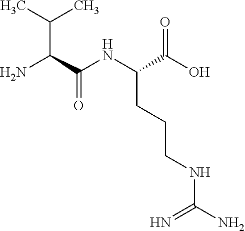 [SMILES: CC(C)[C@H](N)C(=O)N[C@@H](CCCNC(=N)N)C(=O)O] |
| 14 | H-Ala-Arg-OH (acetate salt) | Hydrophobic (link to amino end of Arg) | 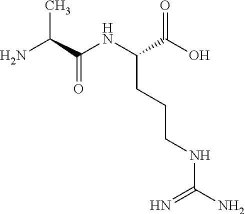 [SMILES: C[C@H](N)C(=O)N[C@@H](CCCNC(=N)N)C(=O)O] |
| 15 | Arg-Arg-Arg-Arg (acetate salt) | Oligopeptide | 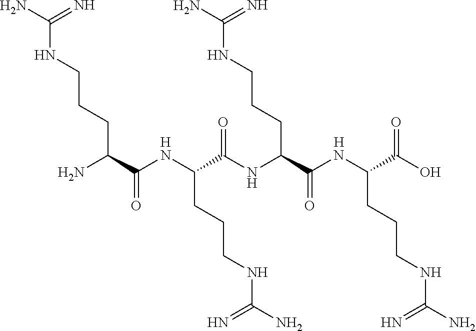 |

Examples of antibodies suitable for the methods and pharmaceutical formulations of the invention include, without limitation, the antibodies listed in Table 2 hereinafter as well as Abagovomab, Abciximab, Actoxumab, Adalimumab, Afelimomab, Afutuzumab, Alacizumab, Alacizumab pegol, ALD518, Alemtuzumab, Alirocumab, Alemtuzumab, Altumomab, Amatuximab, Anatumomab mafenatox, Anrukinzumab, Apolizumab, Arcitumomab, Aselizumab, Altinumab, Atlizumab, Atorolimiumab, tocilizumab, Bapineuzumab, Basiliximab, Bavituximab, Bectumomab, Belimumab, Benralizumab, Bertilimumab, Besilesomab, Bevacizumab, Bezlotoxumab, Biciromab, Bivatuzumab, Bivatuzumab mertansine, Blinatumomab, Blosozumab, Brentuximab vedotin, Briakinumab, Brodalumab, Canakinumab, Cantuzumab mertansine, Cantuzumab mertansine, Caplacizumab, Capromab pendetide, Carlumab, Catumaxomab, CC49, Cedelizumab, Certolizumab pegol, Cetuximab, Citatuzumab bogatox, Cixutumumab, Clazakizumab, Clenoliximab, Clivatuzumab tetraxetan, Conatumumab, Crenezumab, CR6261, Dacetuzumab, Daclizumab, Dalotuzumab, Daratumumab, Demcizumab, Denosumab, Detumomab, Dorlimomab aritox, Drozitumab, Duligotumab, Dupilumab, Ecromeximab, Eculizumab, Edobacomab, Edrecolomab, Efalizumab, Efungumab, Elotuzumab, Elsilimomab, Enavatuzumab, Enlimomab pegol, Enokizumab, Enokizumab, Enoticumab, Enoticumab, Ensituximab, Epitumomab cituxetan, Epratuzumab, Erenumab, Erlizumab, Ertumaxomab, Etaracizumab, Etrolizumab, Evolocumab, Exbivirumab, Exbivirumab, Fanolesomab, Faralimomab, Farletuzumab, Fasinumab, FBTA05, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, GS6624, Ibalizumab, Ibritumomab tiuxetan, Icrucumab, Igovomab, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lebrikizumab, Lemalesomab, Lerdelimumab, Lexatumumab, Libivirumab, Ligelizumab, Lintuzumab, Lirilumab, Lorvotuzumab mertansine, Lucatumumab, Lumiliximab, Mapatumumab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD3, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Ocaratuzumab, Ocrelizumab, Odulimomab, Ofatumumab, Olaratumab, Olokizumab, Omalizumab, Onartuzumab, Oportuzumab monatox, Oregovomab, Orticumab, Otelixizumab, Oxelumab, Ozanezumab, Ozoralizumab, Pagibaximab, Palivizumab, Panitumumab, Panobacumab, Parsatuzumab, Pascolizumab, Pateclizumab, Patritumab, Pemtumomab, Perakizumab, Pertuzumab, Pexelizumab, Pidilizumab, Pintumomab, Placulumab, Ponezumab, Prezalumab, Priliximab, Pritumumab, PRO 140, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Sevirumab, Sibrotuzumab, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Tefibazumab, Telimomab aritox, Tenatumomab, Teneliximab, Teplizumab, Teprotumumab, Tezepelumab, TGN1412, Tremelimumab, Ticilimumab, Tildrakizumab, Tigatuzumab, TNX-650, Tocilizumab, Toralizumab, Tositumomab, Tralokinumab, Trastuzumab, TRBSO7, Tregalizumab, Tremelimumab, Tucotuzumab celmoleukin, Tuvirumab, Ublituximab, Urelumab, Urtoxazumab, Ustekinumab, Vapaliximab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab and Zolimomab aritox.

Intact antibodies are preferred for use in the formulations and methods of the present invention. Most preferred antibodies for use in the formulations and methods of the present invention are adalimumab, bevacizumab, blinatumomab, cetuximab, conatumumab, denosumab, eculizumab, erenumab, evolocumab, infliximab, natalizumab, panitumumab, prezalumab, rilotumumab, rituximab, romosozumab, tezepelumab, and trastuzumab, and antibodies selected from Table 2.

TABLE 2
Preferred Antibodies

| | | | HC Type | | | LC | HC |
|---|---|---|---|---|---|---|---|
| Target | Conc, | Visc, | (including | | | SEQ ID | SEQ ID |
| (informal name) | mg/ml | cP | allotypes) | LC Type | pI | NO | NO |
| anti-amyloid | 142.2 | 5.0 | IgG1 (f) | Kappa | 9.0 | 18 | 19 |
| | | | (R; EM) | | | | |
| GMCSF (247) | 139.7 | 5.6 | IgG2 | Kappa | 8.7 | 20 | 21 |
| CGRPR | 136.6 | 6.3 | IgG2 | Lambda | 8.6 | 22 | 23 |
| RANKL | 152.7 | 6.6 | IgG2 | Kappa | 8.6 | 24 | 25 |
| Sclerostin | 145.0 | 6.7 | IgG2 | Kappa | 6.6 | 26 | 27 |
| (27H6) | | | | | | | |
| IL-1R1 | 153.9 | 6.7 | IgG2 | Kappa | 7.4 | 28 | 29 |
| Myostatin | 141.0 | 6.8 | IgG1 (z) | Kappa | 8.7 | 30 | 31 |
| | | | (K; EM) | | | | |
| B7RP1 | 137.5 | 7.7 | IgG2 | Kappa | 7.7 | 32 | 33 |
| Amyloid | 140.6 | 8.2 | IgG1 (za) | Kappa | 8.7 | 34 | 35 |
| | | | (K; DL) | | | | |
| GMCSF (3.112) | 156.0 | 8.2 | IgG2 | Kappa | 8.8 | 36 | 37 |
| CGRP (32H7) | 159.5 | 8.3 | IgG2 | Kappa | 8.7 | 38 | 39 |
| CGRP (3B6.2) | 161.1 | 8.4 | IgG2 | Lambda | 8.6 | 40 | 41 |
| PCSK9 (8A3.1) | 150.0 | 9.1 | IgG2 | Kappa | 6.7 | 42 | 43 |
| PCSK9 (492) | 150.0 | 9.2 | IgG2 | Kappa | 6.9 | 44 | 45 |
| CGRP | 155.2 | 9.6 | IgG2 | Lambda | 8.8 | 46 | 47 |
| Hepcidin | 147.1 | 9.9 | IgG2 | Lambda | 7.3 | 48 | 49 |
| TNFR p55) | 157.0 | 10.0 | IgG2 | Kappa | 8.2 | 50 | 51 |
| OX40L | 144.5 | 10.0 | IgG2 | Kappa | 8.7 | 52 | 53 |
| HGF | 155.8 | 10.6 | IgG2 | Kappa | 8.1 | 54 | 55 |
| GMCSF | 162.5 | 11.0 | IgG2 | Kappa | 8.1 | 56 | 57 |
| Glucagon R | 146.0 | 12.1 | IgG2 | Kappa | 8.4 | 58 | 59 |
| GMCSF (4.381) | 144.5 | 12.1 | IgG2 | Kappa | 8.4 | 60 | 61 |
| Sclerostin | 155.0 | 12.1 | IgG2 | Kappa | 7.8 | 62 | 63 |
| (13F3) | | | | | | | |
| CD-22 | 143.7 | 12.2 | IgG1 (f) | Kappa | 8.8 | 64 | 65 |
| | | | (R; EM) | | | | |
| INFgR | 154.2 | 12.2 | IgG1 (za) | Kappa | 8.8 | 66 | 67 |
| | | | (K; DL) | | | | |
| Ang2 | 151.5 | 12.4 | IgG2 | Kappa | 7.4 | 68 | 69 |
| TRAILR2 | 158.3 | 12.5 | IgG1 (f) | Kappa | 8.7 | 70 | 71 |
| | | | (R; EM) | | | | |
| EGFR | 141.7 | 14.0 | IgG2 | Kappa | 6.8 | 72 | 73 |
| IL-4R | 145.8 | 15.2 | IgG2 | Kappa | 8.6 | 74 | 75 |
| IL-15 | 149.0 | 16.3 | IgG1 (f) | Kappa | 8.8 | 76 | 77 |
| | | | (R; EM) | | | | |
| IGF1R | 159.2 | 17.3 | IgG1 (za) | Kappa | 8.6 | 78 | 79 |
| | | | (K; DL) | | | | |
| IL-17R | 150.9 | 19.1 | IgG2 | Kappa | 8.6 | 80 | 81 |
| Dkk1 (6.37.5) | 159.4 | 19.6 | IgG2 | Kappa | 8.2 | 82 | 83 |
| Sclerostin | 134.8 | 20.9 | IgG2 | Kappa | 7.4 | 84 | 85 |
| TSLP | 134.2 | 21.4 | IgG2 | Lambda | 7.2 | 86 | 87 |
| Dkk1 (11H10) | 145.3 | 22.5 | IgG2 | Kappa | 8.2 | 88 | 89 |
| PCSK9 | 145.2 | 22.8 | IgG2 | Lambda | 8.1 | 90 | 91 |
| GIPR | 150.0 | 23.0 | IgG1 (z) | Kappa | 8.1 | 92 | 93 |
| (2G10.006) | | | (K; EM) | | | | |
| Activin | 133.9 | 29.4 | IgG2 | Lambda | 7.0 | 94 | 95 |
| Sclerostin (2B8) | 150.0 | 30.0 | IgG2 | Lambda | 6.7 | 96 | 97 |
| Sclerostin | 141.4 | 30.4 | IgG2 | Kappa | 6.8 | 98 | 99 |
| c-fms | 146.9 | 32.1 | IgG2 | Kappa | 6.6 | 100 | 101 |
| α4β7 | 154.9 | 32.7 | IgG2 | Kappa | 6.5 | 102 | 103 |

In the foregoing Table 2 and throughout, “LC” refers to the light chain of an antibody and “HC” refers to the antibody's heavy chain.

### Protein Formulation Preparation

The formulations of the present invention can be prepared by either of three processes designated processes 1, 2, and 3 hereinafter. Process 1 is a method for preparing a formulation of the present invention comprising:
a. dialyzing and/or concentrating a solution comprising a therapeutic protein against a dialysis solution and
b. adding an excipient solution or dry excipient mixture into the protein solution at a selected pH to achieve a desired final excipient concentration, a desired protein concentration, and a desired pH.

Process 2 is a method for preparing a formulation of the present invention comprising:
a. dialyzing a solution of therapeutic protein against a dialysis solution;
b. adding an excipient solution or dry excipient mixture into said dialyzed protein solution at a selected pH and a desired excipient concentration, and
c. concentrating the solution resulting from step b to a desired protein concentration and a desired pH.

Process 3 is a method of preparing a formulation of the present invention comprising:
a. dialyzing a solution comprising a therapeutic protein against a dialysis solution, wherein the dialysis solution comprises the oligopeptide, and
b. optionally concentrating to a desired protein concentration and a desired pH.

In processes 1, 2, and 3, the pH of the concentrated protein can range from about 4.5 to about 6.5. Each excipient can be added before or after the protein concentration step by addition of a concentrated excipient solution or dry excipient or dialysis to achieve the desired excipient and protein concentration. Where a particular excipient is reported in a formulation by, for example, percent (%) w/v, those skilled in the art will recognize that the equivalent molar concentration of that excipient is also contemplated.

The example protein tested was an IgG2 monoclonal antibody (mAb) formulated with an acetate buffer at a buffer concentration (e.g., 10 mM) resulting from prior processing, with the acetate level allowed to change to attain a charge neutral formulation at the desired pH and protein concentration. Preferably, the formulation comprises polysorbate 20 or polysorbate 80. The pH range of the prepared solutions was 5-6 that was not further adjusted except for those specified in the Example 5. The protein concentration of each sample was measured using SoloVPE UV spectroscopy. The samples were stored at 2-8° C. until being brought to room temperature prior to sample loading on the viscometer. The samples were measured within 2 weeks of preparation (usually within 2-3 days).

### Viscosity Measurement

The viscosity of the protein formulations was measured using a standard cone-and-plate rotational viscometer (Malvern Instruments Gemini 200 Rheometer using a 40 mm diameter with 1 degree cone, and water solvent trap) at a temperature 25° C. and a shear rate range of 100-1000 S⁻¹. Upon loading, each sample was allowed to equilibrate for 2 minutes at 25° C. prior to the start of data collection. All formulation samples tested showed Newtonian rheological behavior. Therefore, the viscosity values reported herein were average values at a shear rate range of 100-1000 S-1.

### WORKING EXAMPLES

### Example 1

### Comparison of Viscosity and Percent Viscosity Reduction Between the Protein Formulations Containing Dipeptide Excipients and Single Amino Acid Excipients

This example illustrates the effect of various types of arginine-containing dipeptides on the viscosity of the protein formulations, and compares viscosity reduction with single amino acid excipients.

An IgG2 antibody (tezepelumab, 128 mg/mL) was prepared by dialyzing the antibody solution against 10 mM acetate buffer (sodium form) at pH 4.3 and then concentrating the dialyzed protein solution by centrifugation to a higher protein concentration. To the concentrated protein solution, certain volumes of a concentrated dipeptide excipient stock solution in 10 mM acetate buffer (sodium form) at pH 5-6 and a 1% polysorbate 20 solution were added to achieve the desired final concentrations of dipeptide excipient (200 mM), polysorbate 20 (0.01% w/v) and protein (128 mg/mL), and pH 5-6. The viscosity data given in Example 1 (Table 3) demonstrated that the arginine-containing dipeptides effectively reduce the viscosity of the protein formulations. At 200 mM excipient concentration, the viscosity reductions of the protein formulations containing the dipeptide excipients are greater than those containing a single amino acid or a mixture of two single amino acids.

TABLE 3
Comparison of viscosity and
percent viscosity reduction between
the protein formulations (128 mg/mL
protein concentration) containing dipeptide
excipients and single amino acid excipients

| | | | Viscosity |
|---|---|---|---|
| | Excipient | Viscosity | reduction |
| Excipient | concentration | (cP) | (relative to the |
| added | (mM) | at 25° C. | control) |
| None (control) | 0 | 10.8 | NA |
| Arg (HCl salt) | 200 | 7.7 | 29% |
| Pro | 200 | 9.2 | 15% |
| Pro + Arg | 100 + 100 | 7.0 | 35% |
| (HCl salt) | | | |
| NAR + Arg | 100 + 100 | 6.8 | 37% |
| (HCl salt) | | | |
| H-Arg-Arg-OH | 200 | 5.5 | 49% |
| (acetate salt) | | | |
| H-Arg-Lys-OH | 200 | 5.7 | 47% |
| (acetate salt) | | | |
| H-Arg-Phe-OH | 200 | 5.4 | 50% |
| (acetate salt) | | | |
| H-Arg-Pro-OH | 200 | 7.4 | 32% |
| (sulfate salt) | | | |
| H-Arg-Val-OH | 200 | 5.3 | 51% |
| (acetate salt) | | | |
| H-Arg-Ala-OH | 200 | 5.8 | 47% |
| (acetate salt) | | | |
| H-Asp-Arg-OH | 200 | 7.1 | 34% |
| (acetate salt) | | | |
| H-Lys-Arg-OH | 200 | 5.2 | 52% |
| (acetate salt) | | | |
| H-Pro-Arg-OH | 200 | 5.9 | 46% |
| (acetate salt) | | | |
| H-Leu-Arg-OH | 200 | 5.5 | 49% |
| (acetate salt) | | | |
| H-Val-Arg-OH | 200 | 5.7 | 47% |
| (acetate salt) | | | |
| H-Ala-Arg-OH | 200 | 5.7 | 47% |
| (acetate salt) | | | |

NAR: N-acetyl arginine.

### Example 2

Effect of Excipient Concentration on Viscosity and Percent Viscosity Reduction of the Protein Formulations at 128 mg/mL Protein Concentration

This example illustrates the effect of various arginine-containing dipeptides at different excipient concentrations on the viscosity of the protein formulations with 128 mg/ml tezepelumab.

The IgG2 antibody was prepared by dialyzing the antibody solution (128 mg/ml) against 10 mM acetate buffer (sodium form) at pH 4.3 and then concentrating the dialyzed protein solution using centrifugation to a higher protein concentration. To the concentrated protein solution, certain volumes of a concentrated dipeptide excipient stock solution in 10 mM acetate buffer (sodium form) at pH 5-6 and a 1% polysorbate 20 solution were added to achieve the desired final concentrations of dipeptide excipient (10-500 mM), polysorbate 20 (0.01% w/v) and protein (128 mg/mL), and pH 5-6. The viscosity data given in Example 2 (Table 4) demonstrated that arginine-containing dipeptides reduce the viscosity of the protein formulations at the excipient concentration range of 10-500 mM. The excipient concentrations at 100-200 mM appear to be the most effective at viscosity reduction.

TABLE 4
Effect of excipient concentration on viscosity and percent viscosity
reduction of the protein formulations (128 mg/mL protein concentration)

| | Excipient | Viscosity | Viscosity reduction |
|---|---|---|---|
| Excipient | concentration | (cP) at | (relative to the |
| added | (mM) | 25° C. | control) |
| None (control) | 0 | 10.8 | NA |
| H-Arg-Arg-OH | 100 | 5.9 | 46% |
| (acetate salt) | 200 | 5.5 | 49% |
| H-Arg-Lys-OH | 100 | 5.8 | 46% |
| (acetate salt) | 200 | 5.7 | 47% |
| H-Arg-Phe-OH | 10 | 10.0 | 7% |
| (acetate salt) | 100 | 6.1 | 44% |
| | 200 | 5.4 | 50% |
| | 500 | 6.1 | 43% |
| H-Arg-Ala-OH | 100 | 6.2 | 43% |
| (acetate salt) | 200 | 5.8 | 47% |
| H-Lys-Arg-OH | 10 | 9.6 | 11% |
| (acetate salt) | 200 | 5.2 | 52% |
| | 500 | 6.9 | 36% |
| H-Pro-Arg-OH | 10 | 11.0 | 0% |
| (acetate salt) | 100 | 7.0 | 35% |
| | 200 | 5.9 | 46% |
| | 500 | 5.5 | 49% |

### Example 3

### Effect of Oligopeptide on the Viscosity of the Protein Formulation at 128 Mg/mL Protein Concentration

This example illustrates the effect of arginine-containing oligomer on the viscosity of the protein formulations.

An IgG2 antibody (tezepelumab, 128 mg/mL) was prepared by dialyzing the antibody solution against 10 mM acetate buffer (sodium form) at pH 4.3 and then concentrating the dialyzed protein solution by centrifugation to a higher protein concentration. To the concentrated protein solution, certain volumes of a concentrated excipient stock solution in 10 mM acetate buffer (sodium form) at pH 5.2 and a 1% polysorbate 20 solution were added to achieve the desired final concentrations of excipient (100 mM), polysorbate 20 (0.01% w/v) and protein (128 mg/mL), and pH 5.2. The viscosity data given in this Example 3 (Table 5) demonstrated that arginine-containing oligomer effectively reduces the viscosity of the protein formulation.

TABLE 5
Effect of oligopeptide on viscosity and percent viscosity reduction
of the protein formulations (128 mg/mL protein concentration)

| | Excipient | | Viscosity reduction |
|---|---|---|---|
| | concentration | Viscosity (cP) | (relative to the |
| Excipient added | (mM) | at 25° C. | control) |
| None (control) | 0 | 10.8 | NA |
| Arg-Arg-Arg-Arg | 100 | 5.1 | 53% |
| (acetate salt) | | | |

### Example 4

### Effect of Dipeptide Excipients on the Viscosity of Protein Formulations at 85 Mg/Ml Protein Concentration

This example illustrates the effect of various types of arginine-containing dipeptides at different excipient concentrations on the viscosity of the protein formulations with 85 mg/ml protein concentration. An IgG2 antibody (tezepelumab, 85 mg/mL) was prepared by dialyzing the antibody solution against 10 mM acetate buffer (sodium form) at pH 5.2 and then concentrating the dialyzed protein solution by centrifugation to a higher protein concentration. To the concentrated protein solution, certain volumes of a concentrated dipeptide excipient stock solution in 10 mM acetate buffer (sodium) at pH 5-6 and a 1% polysorbate 20 solution were added to achieve the desired final concentrations of dipeptide excipient (100 mM or 200 mM), polysorbate 20 (0.01% w/v) and protein (85 mg/mL) and pH 5-6. The viscosity data given in Example 4 (Table 6) demonstrated that arginine-containing dipeptides also reduce the viscosity of the protein formulations with 85 mg/ml protein. The viscosity reductions of the protein formulations containing the dipeptide excipients are greater than those containing a single amino acid at the same excipient concentration.

TABLE 6
Viscosity and percent viscosity reduction of protein
formulations at 85 mg/mL protein concentration formulated
with dipeptide excipients at different excipient concentrations

| | Excipient | Viscosity | Viscosity reduction |
|---|---|---|---|
| Excipient | concentration | (cP) at | (relative to the |
| added | (mM) | 25° C. | control) |
| None (control) | 0 | 4.0 | NA |
| Arg (HCl) | 100 | 3.1 | 21% |
| | 200 | 3.0 | 25% |
| NAR | 100 | 3.2 | 19% |
| H-Arg-Arg-OH | 100 | 2.6 | 35% |
| (acetate salt) | 200 | 2.7 | 32% |
| H-Arg-Lys-OH | 100 | 2.5 | 36% |
| (acetate salt) | 200 | 2.7 | 31% |
| H-Arg-Phe-OH | 100 | 2.7 | 31% |
| (acetate salt) | 200 | 2.7 | 32% |
| H-Arg-Ala-OH | 100 | 2.6 | 34% |
| (acetate salt) | 200 | 2.6 | 34% |
| H-Pro-Arg-OH | 100 | 2.6 | 34% |
| (acetate salt) | | | |

NAR: N-acetyl-Arginine.

### Example 5

### Effect of pH on the Viscosity Reduction of the Protein Formulations

This example illustrates the effect of solution pH on the viscosity reduction of the protein formulations. An IgG2 antibody (tezepelumab, 130 mg/mL) was prepared by dialyzing the antibody solution against 10 mM acetate buffer (sodium form), pH 4.3 and then concentrating the dialyzed protein solution using a centrifugation method to a higher protein concentration.

To the concentrated protein solution, the certain volumes of a concentrated dipeptide excipient stock solution in 10 mM acetate buffer (sodium form) pH 5 or pH 6 and a 1% polysorbate 20 solution were added to achieve the desired final concentrations of dipeptide excipient (100 mM or 200 mM), polysorbate 20 (0.01% w/v) and protein (130 mg/mL) and pH 5 or pH 6. The viscosity data given in Example 5 (Table 7) demonstrated that arginine-containing dipeptide excipient effectively reduce the viscosity of the protein formulation at the tested pH range.

TABLE 7
Effect of solution pH on viscosity
reduction of the protein formulations

| | Excipient | | Viscosity reduction |
|---|---|---|---|
| Excipient added | concentration (mM) | pH | (relative to the control) |
| H-Arg-Phe-OH | 100 | 5 | 44% |
| (acetate salt) | | | |
| H-Arg-Phe-OH | 100 | 6 | 55% |
| (acetate salt) | | | |
| H-Arg-Phe-OH | 200 | 5 | 50% |
| (acetate salt) | | | |
| H-Arg-Phe-OH | 200 | 6 | 58% |
| (acetate salt) | | | |

### Example 6

### Effect of Dipeptide Excipient on Viscosity Reduction of the Protein Formulation as a Function of Protein and Excipient Concentrations

This example illustrates the effect of the arginine-containing dipeptide (Arg-Phe) on viscosity reduction of the protein formulation as a function of protein and excipient concentrations.

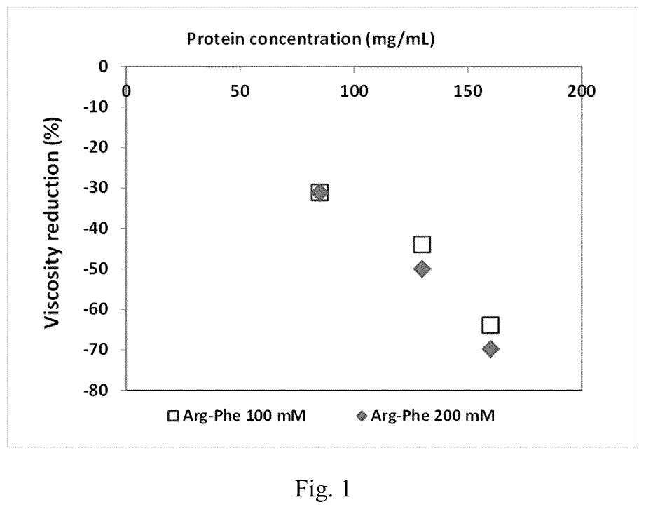

A solution of tezepelumab containing 85 mg/mL or 128 mg/mL or 160 mg/mL protein concentration with 10 mM acetate buffer (based on prior formulation processing) and 0.01% polysorbate 20) was formulated with Arg-Phe excipient at 100 mM or 200 mM excipient concentration, at pH 5. The viscosity data given in FIG. 1 demonstrated that percentage of viscosity reduction increased with increased protein concentration.

### Example 7

### Effect of Various Dipeptide Excipients on Viscosity Reduction of Another IgG2 mAb Formulations

This example illustrates the effect of various arginine-containing dipeptides and a mixture composition of the dipeptide and single amino acid on the viscosity reduction of another mAb's formulations.

The example protein tested was evolocumab (PCSK9 binding polypeptide, IgG2 mAb). The formulations of the present invention were prepared by dialyzing an antibody solution against 10 mM of sodium acetate buffer pH 4 and then concentrating the protein solution using centrifugation to a desired protein concentration (150 mg/mL) followed by addition of a polysorbate 80 stock solution to achieve a desired final concentration of polysorbate (0.01% w/v). To a given volume of each concentrated protein solution, the weighted amount of each dipeptide excipient powder was added to achieve the desired final dipeptide excipient concentration (150 mM for each dipeptide excipient or 140 mM dipeptide plus 50 mM single amino acid for a mixture composition) (Table 8), and a final pH range was 5-6.

The viscosity data given in this example 7 (Table 8) demonstrated that all arginine-containing dipeptides or a mixture composition of dipeptide and single amino acid tested effectively reduce the viscosity of the evolocumab formulations. The viscosity reduction range was about from 61-76% relative to the control at the same protein concentration (150 mg/mL).

TABLE 8
Effect of the various excipients on viscosity and percent viscosity
reduction of evolocumab formulations (protein concertation 150 mg/mL)

| | Excipient | Viscosity (cP) | Viscosity reduction |
|---|---|---|---|
| Excipient added | concentration (mM) | at 25° C. | (relative to the control) |
| None (control) | 0 | 39.6 | NA |
| H-Arg-Arg-OH | 150 | 12.5 | 68% |
| (acetate salt) | | | |
| H-Arg-Phe-OH | 150 | 10.7 | 73% |
| (acetate salt) | | | |
| H-Arg-Pro-OH | 150 | 13.9 | 65% |
| (acetate salt) | | | |
| H-Arg-Tyr-OH | 150 | 13.0 | 67% |
| (acetate salt | | | |
| H-Arg-Ala-OH | 150 | 14.9 | 62% |
| (acetate salt) | | | |
| H-Arg-Val-OH | 150 | 15.3 | 61% |
| (acetate salt) | | | |
| H-Phe-Arg-OH | 150 | 10.0 | 75% |
| (acetate salt) | | | |
| H-Pro-Arg-OH | 150 | 10.0 | 75% |
| (acetate salt) | | | |
| H-Val-Arg-OH | 150 | 11.8 | 70% |
| (acetate salt) | | | |
| H-Ala-Arg-OH | 150 | 12.8 | 68% |
| (acetate salt) | | | |
| H-Arg-Phe-OH | 140 + 50 | 9.8 | 75% |
| (acetate) + Arg (HCl) | | | |
| H-Pro-Arg-OH | 140 + 50 | 9.5 | 76% |
| (acetate) +Arg (HCl) | | | |

### Example 8

### Dipeptide Stability Studies

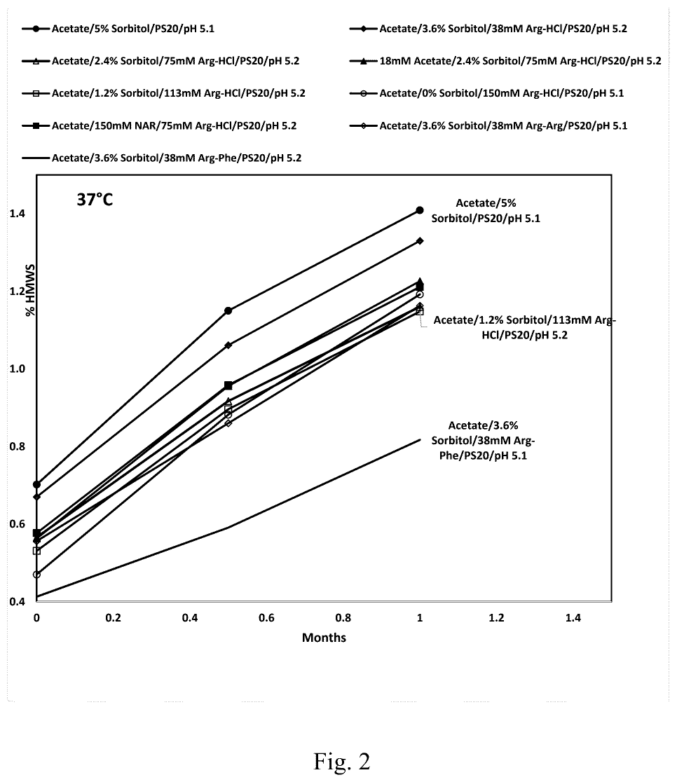

Two dipeptides consisting of arginine-arginine (Arg-Arg) and arginine-phenylalanine (Arg-Phe) were evaluated with denosumab at 120 mg/mL. Each formulation consisted of approximately 10 mM acetate, 3.6% (w/v) sorbitol, and 0.01% (w/v) polysorbate 20 at a final pH of 5.1 containing either 38 mM of Arg-Arg or Arg-Phe. The formulations were filled into glass vials at a fill volume of 1.0 mL. The formulations were stored at temperatures of 2° C. to 8° C. for up to 12 months and 25° C., 30° C., and 37° C. for 3 months. The stability as based on formation of HMWS was assessed using SE-HPLC. The stability of these dipeptide formulations were compared with Arg-HCl formulations at 37° C. as shown in FIG. 2. The formulation compositions and a key to the abbreviations in FIG. 2 appear in Table 9 below.

TABLE 9
Formulations used in Stability Studies

| Abbreviation | Formulation Composition |
|---|---|
| Acetate/5% | 10 mM Acetate, 5% (w/v) Sorbitol, |
| Sorbitol/ | 0.01% (w/v) Polysorbate 20, pH 5.1 |
| PS20/pH 5.1 | |
| Acetate/3.6% | 10 mM Acetate, 3.6% (w/v) Sorbitol, |
| Sorbitol/38 | 0.01% (w/v) Polysorbate 20, 38 mM |
| mM Arg-HCl/ | Arginine HCl, pH 5.2 |
| PS20/pH 5.2 | |
| Acetate/2.4% | 10 mM Acetate, 2.4% (w/v) Sorbitol, |
| Sorbitol/75 | 0.01% (w/v) Polysorbate 20, 75 mM |
| mM Arg-HCl/ | Arginine HCl, pH 5.2 |
| PS20/pH 5.2 | |
| 18 mM | 18 mM Acetate, 2.4% (w/v) Sorbitol, |
| Acetate/2.4% | 0.01% (w/v) Polysorbate 20, 75 mM |
| Sorbitol/75 | Arginine HCl, pH 5.2 |
| mM Arg-HCl/ | |
| PS20/pH 5.2 | |
| Acetate/1.2% | 10 mM Acetate, 1.2% Sorbitol, 0.01% |
| Sorbitol/113 | (w/v) Polysorbate 20, 113 mM Arginine |
| mM Arg-HCl/ | HCl, pH 5.2 |
| PS20/pH 5.2 | |
| Acetate/0% | 10 mM Acetate, 0% (w/v) sorbitol, 0.01% |
| Sorbitol/150 | (w/v) Polysorbate 20, 150 mM Arginine |
| mM Arg-HCl/ | HCl, pH 5.1 |
| PS20/pH 5.1 | |
| Acetate/150 | 10 mM Acetate, 75 mM Arginine HCl, |
| mM NAR/75 | 150 mM NAR, 0.01% (w/v) Polysorbate |
| mM Arg-HCl/ | 20, pH 5.1 |
| PS20/pH 5.2 | |
| Acetate/3.6% | 10 mM Acetate, 3.6% (w/v) Sorbitol, |
| Sorbitol/38 | 0.01% (w/v) Polysorbate 20, 38 mM |
| mM Arg-Arg/ | Arginine-Arginine, pH 5.1 |
| PS20/pH 5.1 | |
| Acetate/3.6% | 10 mM Acetate, 3.6% (w/v) Sorbitol, |
| Sorbitol/38 | 0.01% (w/v) Polysorbate 20, 38 mM |
| mM Arg-Phe/ | Arginine-Phenylalanine, pH 5.2 |
| PS20/pH 5.2 | |

### Example 9

Effect of Various Dipeptide Excipients on Viscosity Reduction of Another IgG2 mAb Formulations at 205-212 mg/mL Protein Concentration

This example illustrates the effect of various arginine-containing dipeptides on the viscosity reduction of another monoclonal antibody's formulations at 205-212 mg/mL protein concentration. This example supports having an antibody in the formulation of the present invention in a concentration of at least about 200 mg/mL.

The example protein tested was evolocumab (PCSK9 binding polypeptide, IgG2 mAb). The antibody solution (120 mg/mL) was dialyzed against 20 mM of sodium acetate buffer pH 4. To a given volume of each dialyzed solution, the weighted amount of each dipeptide excipient powder was added to achieve the desired final dipeptide excipient concentration (120 mM for each dipeptide excipient, Table 10 below). Each formulation solution was concentrated using centrifugation to a desired protein concentration (205 mg/mL or 212 mg/mL) followed by addition of a polysorbate 80 stock solution to achieve a desired final concentration of polysorbate (0.01% w/v). The final pH of each formulation was 5.4.

The viscosity data given in this example 9 (Table 10) demonstrated that all arginine-containing dipeptides tested effectively reduce the viscosity of the evolocumab formulations. The viscosity values of the evolocumab formulations containing arginine dipeptides at 205 mg/mL or 212 mg/mL protein concentration are close to the viscosity of the formulation without dipeptide excipient at 150 mg/mL protein concentration.

TABLE 10
Effect of the various excipients on viscosity reduction of evolocumab
formulations (protein concertation 205 mg/mL or 212 mg/mL

| | Protein | Excipient | Viscosity |
|---|---|---|---|
| | Concentration | concentration | (cP) at |
| Excipient added | (mg/mL) | (mM) | 25° C. |
| None (control) | 150 | 0 | 39.6 |
| H-Arg-Phe-OH | 205 | 120 | 40.5 |
| (acetate salt) | | | |
| H-Phe-Arg-OH | 212 | 120 | 38.2 |
| (acetate salt) | | | |
| H-Arg-Pro-OH | 205 | 120 | 40.6 |
| (acetate salt) | | | |
| H-Pro-Arg-OH | 205 | 120 | 39.0 |
| (acetate salt) | | | |
| H-Val-Arg-OH | 205 | 120 | 47.3 |
| (acetate salt) | | | |

The inventions of this specification relate to the following embodiments:
1. A liquid pharmaceutical formulation comprising an antibody, a buffer, and at least one oligopeptide, wherein (i) the oligopeptide comprises an arginine residue and consists of 2 to 10 amino acid residues but is not arg-asp, arg-glu, or poly-arginine, (ii) the buffer is acetate, glutamate, or phosphate and (iii) the pH of the formulation is about 4.5 to about 6.5.
2. The formulation of embodiment 1, wherein the antibody is present in a concentration of at least about 70 mg/mL.
3. The formulation of embodiment 1, wherein the antibody is present in a concentration of at least about 85 mg/mL.
4. The formulation of embodiment 1, wherein the antibody is present in a concentration of at least about 100 mg/mL.
5. The formulation of embodiment 1, wherein the antibody is present in a concentration of at least about 130 mg/mL.
6. The formulation of embodiment 1, wherein the antibody is present in a concentration of at least about 160 mg/mL.
7. The formulation of embodiment 1, wherein the antibody is present in a concentration of at least about 200 mg/mL
8. The formulation of embodiment 1, wherein the antibody is selected from adalimumab, bevacizumab, blinatumomab, cetuximab, conatumumab, denosumab, eculizumab, erenumab, evolocumab, infliximab, natalizumab, panitumumab, prezalumab, rilotumumab, rituximab, romosozumab, tezepelumab, and trastuzumab, or comprises the heavy chain and light chain sequences of an antibody selected from Table 2.
9. The formulation of embodiment 1, wherein the antibody is tezepelumab or a PCSK9 binding polypeptide.
10. The formulation of embodiment 1, wherein the antibody is evolocumab.
11. The formulation of embodiment 1, wherein the oligopeptide comprises an arginine residue at its N-terminus.
12. The formulation of embodiment 1, wherein the oligopeptide comprises an arginine residue at its C-terminus.
13. The formulation of embodiment 1, wherein the oligopeptide comprises more than one arginine residue.
14. The formulation of embodiment 1, wherein the oligopeptide has a concentration of about 10 mM to about 500 mM.
15. The formulation of embodiment 1, wherein the oligopeptide has a concentration of about 100 mM to about 200 mM.
16. The formulation of embodiment 1, wherein the oligopeptide is an acetate salt.
17. The formulation of embodiment 1, wherein the oligopeptide is a dipeptide comprising a basic, acidic, hydrophobic, hydrophilic, or aromatic residue.
18. The formulation of embodiment 1, wherein the oligopeptide is selected from Arg-Lys, Arg-Phe, Arg-Pro, Arg-Val, Arg-Ala, Asp-Arg, Lys-Arg, Pro-Arg, Leu-Arg, Val-Arg, Phe-Arg, Arg-Tyr, and Ala-Arg,.
19. The formulation of embodiment 1, wherein the oligopeptide is selected from Pro-Arg, Phe-Arg, Arg-Phe, Arg-Val, Val-Arg, Arg-Tyr, and Lys-Arg.
20. The formulation of embodiment 1, wherein the buffer is acetate.
21. The formulation of embodiment 19, wherein the acetate buffer has a concentration of about 10 mM.
22. The formulation of embodiment 1, wherein the formulation has a pH of about 5 to about 6.
23. The formulation of embodiment 1, further comprising a surfactant.
24. The formulation of embodiment 23, wherein the surfactant is polysorbate 20 or polysorbate 80.
25. The formulation of embodiment 23, wherein the surfactant is 0.01% polysorbate 20.
26. The formulation of embodiment 23, wherein the surfactant is 0.01% polysorbate 80.
27. The formulation of embodiment 1, further comprising a second oligopeptide comprising arginine and consisting of two to 10 amino acid residues.
28. The formulation of embodiment 27, wherein the second oligopeptide is a dipeptide comprising a basic, acidic, hydrophobic, or aromatic residue.
29. The formulation of embodiment 27, wherein the second oligopeptide is selected from Arg-Lys, Arg-Pro, Arg-Ala, Asp-Arg, Lys-Arg, Pro-Arg, Val-Arg, Ala-Arg, Phe-Arg, and Arg-Tyr.
30. The formulation of embodiment 1, further comprising one or more amino acids.
31. The formulation of embodiment 30, wherein the one or more amino acids are selected from arginine, proline, n-acetyl arginine, n-acetyl lysine, n-acetyl histidine, n-acetyl proline or mixtures of any thereof.
32. A method of reducing viscosity in a pharmaceutical formulation comprising an antibody, wherein the method comprises providing a solution at a pH of about 4.5 to about 6.5 comprising (i) the antibody, (ii) an oligopeptide salt, wherein the oligopeptide comprises an arginine residue, consists of 2 to 10 amino acid residues and is present in a viscosity-reducing concentration, and (iii) a buffer.
33. The method of embodiment 32, wherein the antibody is present in a concentration of at least about 70 mg/mL.
34. The method of embodiment 32, wherein the antibody is present in a concentration of at least about 85 mg/mL.
35. The method of embodiment 32, wherein the antibody is present in a concentration of at least about 100 mg/mL.
36. The method of embodiment 32, wherein the antibody is present in a concentration of at least about 130 mg/mL.
37. The method of embodiment 32, wherein the antibody is present in a concentration of at least about 160 mg/mL.
38. The method of embodiment 32, wherein the antibody is selected from adalimumab, bevacizumab, blinatumomab, cetuximab, conatumumab, denosumab, eculizumab, erenumab, evolocumab, infliximab, natalizumab, panitumumab, rilotumumab, rituximab, romosozumab, tezepelumab, and trastuzumab, or comprises the heavy chain and light chain sequences of an antibody selected from Table 2.
39. The method of embodiment 32, wherein the antibody is tezepelumab or a PCSK9 binding polypeptide.
40. The method of embodiment 32, wherein the antibody is evolocumab.
41. The method of embodiment 32, wherein the oligopeptide comprises an arginine residue at its N-terminus.
42. The method of embodiment 32, wherein the oligopeptide comprises an arginine residue at its C-terminus.
43. The method of embodiment 32, wherein the oligopeptide has a concentration of about 10 mM to about 500 mM.
44. The method of embodiment 32, wherein the oligopeptide has a concentration of about 100 mM to about 200 mM.
45. The method of embodiment 32, wherein the oligopeptide salt is an acetate salt, a sulfate salt, hydrochloride salt or a glutamate salt.
46. The method of embodiment 32, wherein the oligopeptide is a dipeptide comprising a basic, acidic, hydrophobic, or aromatic residue.
47. The method of embodiment 32, wherein the oligopeptide is selected from Arg-Lys, Arg-Phe, Arg-Pro, Arg-Val, Arg-Ala, Asp-Arg, Lys-Arg, Pro-Arg, Leu-Arg, Val-Arg, Phe-Arg, Arg-Tyr, and Ala-Arg.
48. The method of embodiment 32, wherein the oligopeptide is selected from Pro-Arg, Phe-Arg, Arg-Phe, Arg-Val, Val-Arg, and Lys-Arg.
49. The method of embodiment 32, wherein the oligopeptide salt is an acetate salt.
50. The method of embodiment 32, wherein the oligopeptide salt is a lyophilized powder prior to being placed in solution.
51. The method of embodiment 32, wherein the buffer is acetate, glutamate or phosphate.
52. The method of embodiment 51, wherein the buffer is acetate buffer and has a concentration of about 10 mM to about 50 mM.
53. The method of embodiment 32, wherein the solution further comprises a surfactant.
54. The method of embodiment 53, wherein the surfactant is polysorbate 20 or polysorbate 80.
55. The method of embodiment 53, wherein the surfactant is 0.01% polysorbate 20.
56. The method of embodiment 32, wherein the pH is about 5 to about 6.
57. The method of embodiment 32, wherein the solution further comprises a second oligopeptide comprising arginine and consisting of two to 10 amino acid residues.
58. The method of embodiment 57, wherein the second oligopeptide is a dipeptide comprising a basic, acidic, hydrophobic, or aromatic residue.
59. The method of embodiment 57, wherein the second oligopeptide is selected from Arg-Lys, Arg-Phe, Arg-Pro, Arg-Val, Arg-Ala, Asp-Arg, Lys-Arg, Pro-Arg, Leu-Arg, Val-Arg, Ala-Arg, Phe-Arg, and Arg-Tyr.
60. The method of embodiment 32, wherein the solution further comprises one or more amino acids.
61. The method of embodiment 60, wherein the one or more amino acids are selected from arginine, proline, n-acetyl arginine, n-acetyl lysine, n-acetyl histidine, n-acetyl proline or mixtures of any thereof.
62. The method of embodiment 32, wherein viscosity of the formulation is reduced by at least about 30%.
63. The method of embodiment 32, wherein viscosity of the formulation is reduced by at least about 50%.
64. A lyophilized powder comprising an antibody and an oligopeptide comprising arginine and consisting of 2 to 10 amino, wherein the oligopeptide is present at a weight:weight concentration effective to reduce viscosity upon reconstitution with a diluent.
65. The lyophilized powder of embodiment 64 wherein the oligopeptide is about 10 μg per mg of antibody to about 50 μg per mg of antibody.
66. The lyophilized powder of embodiment 64 wherein the oligopeptide is about 50 μg per mg of antibody to about 1 mg per mg of antibody.
67. The lyophilized powder of embodiment 64 wherein the oligopeptide is about 150 μg per mg of antibody to about 250 μg per mg of antibody.
68. The lyophilized powder of embodiment 64 wherein the oligopeptide is about 200 μg to about 500 μg per mg of antibody.
69. The lyophilized powder of embodiment 64 wherein the oligopeptide is about 150 μg to about 250 μg per mg of antibody.
70. A method of reconstituting the lyophilized powder of any of embodiments 64 to 69, which comprises adding a sterile aqueous diluent comprising acetate or glutamate buffer in sufficient concentration so that the reconstituted solution has a pH of about 4 to about 8.
71. The method of embodiment 70, wherein the acetate or glutamate buffer is in sufficient concentration so that the reconstituted solution has a pH of about 4.5 to about 6.5.
72. A pharmaceutical formulation of tezepelumab, which comprises an excipient selected from arginine, proline, proline and arginine, N-acetyl arginine, Arg-Lys, Arg-Phe, Arg-Pro, Arg-Val, Arg-Ala, Asp-Arg, Lys-Arg, Pro-Arg, Leu-Arg, Val-Arg, or Ala-Arg.
73. The formulation of embodiment 72, wherein tezepelumab is present at a concentration of about 85 mg/mL to about 128 mg/mL
74. The formulation of embodiment 72, wherein the pH is about 5-6 and the formulation comprises sodium acetate.
75. The formulation of embodiment 72, wherein the excipient is present in a concentration of about 100 to 200 mM.
76. A pharmaceutical formulation of PCSK9 binding polypeptide, wherein the formulation comprises an excipient selected from Arg-Phe, Arg-Ala, Arg-Val, Pro-Arg, Val-Arg, Ala-Arg, Phe-Arg, Arg-Tyr, and Arg-Phe.
77. The formulation of embodiment 76, further comprising arginine.
78. The formulation of embodiment 76, wherein the PCSK9 polypeptide is present at a concentration of about 150 mg/mL
79. The formulation of embodiment 76, wherein the pH is about 5-6 and the formulation comprises sodium acetate.
80. The formulation of embodiment 76, wherein the excipient is present in a concentration of about 140 to 150 mM.
81. The formulation of embodiment 77, wherein the arginine is present in a concentration of about 50 mM.
82. The formulation of any of embodiments 1-31 and 73-81 further comprising one or more additional excipients selected from stabilizing agents, tonicifying agents, and anti-oxidants.
83. The formulation of embodiment 82, wherein said additional excipients are selected from sucrose, trehalose, sorbitol, mannitol, glucose, lactose, cyclodextrin derivatives, glycine, proline, glutamate, glycerol, urea, EDTA, EGTA, and DTPA.
84. The method of any of embodiments 32-63 wherein the solution further comprises one or more additional excipients selected from stabilizing agents, tonicifying agents, and anti-oxidants.
85. The method of embodiment 84, wherein said additional excipients are selected from sucrose, trehalose, sorbitol, mannitol, glucose, lactose, cyclodextrin derivatives, glycine, proline, glutamate, glycerol, urea, EDTA, EGTA, and DTPA.
86. The lyophilized powder of any of embodiments 64-69, wherein the lyophilized powder further comprises one or more additional excipients selected from stabilizing agents, tonicifying agents, anti-oxidants, and surfactants.
87. The lyophilized powder of embodiment 86, wherein said additional excipients are selected from sucrose, trehalose, sorbitol, mannitol, glucose, lactose, cyclodextrin derivatives, glycine, proline, glutamate, glycerol, urea, EDTA, EGTA, DTPA, polysorbate 20, polysorbate 80, and poloxamer 188.
88. The method of embodiment 70, wherein the lyophilized powder further comprises additional excipients selected from stabilizing agents, tonicifying agents, anti-oxidants, and surfactants.
89. The method of embodiment 88, wherein said additional excipients are selected from sucrose, trehalose, sorbitol, mannitol, glucose, lactose, cyclodextrin derivatives, glycine, proline, glutamate, glycerol, urea, EDTA, EGTA, DTPA, polysorbate 20, polysorbate 80, and poloxamer 188.
90. A method of preparing a pharmaceutical formulation of any of embodiments 1-31, which comprises:
   a. dialyzing and/or concentrating a solution comprising the antibody against a dialysis solution and
   b. adding an excipient solution or dry excipient mixture into the antibody solution at a selected pH to achieve a desired excipient concentration, a desired protein concentration, and a desired pH.
   c. A method of preparing a pharmaceutical formulation of any of embodiments 1-31, which comprises:
   d. dialyzing a solution of the antibody against a dialysis solution;
   e. adding an excipient solution or dry excipient mixture into said dialyzed protein solution at a selected pH and a desired excipient concentration; and
   f. concentrating the solution resulting from step b to a desired protein concentration and desired pH.
91. A method of preparing a pharmaceutical formulation of any of embodiments 1-31, which comprises:
   a. dialyzing a solution of therapeutic protein against a dialysis solution, wherein the dialysis solution comprises the oligopeptide, and
   b. optionally concentrating to a desired protein concentration.
92. The formulation of embodiment 1, wherein the antibody is present in a concentration of about 200 mg/mL to about 400 mg/mL.
93. The method of embodiment 32, wherein the antibody is present in a concentration of at least about 200 mg/mL
94. The method of embodiment 32, wherein the antibody is present in a concentration of about 200 mg/mL to about 400 mg/mL.

All publications, patents, and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the appended embodiments.

Those skilled in the art will recognize, or be able to ascertain using no more than routine experimentation, many equivalents to the specific embodiments of the invention described herein. Such equivalents are intended to be encompassed by the embodiments that follow.

### ABBREVIATIONS

The abbreviations appearing throughout this specification are defined as follows.
CMC carboxymethyl cellulose
DTPA Pentetic acid (diethylenetriaminepentaacetic acid)
EDTA Ethylenediaminetetraacetic acid
EGTA ethylene glycol-bis(β-aminoethyl ether)-N,N,N′,N′-tetraacetic acid
HEC hydroxyethyl cellulose
HMWS high molecular weight species
HSA human serum albumin
NAR N-acetyl arginine
PEG polyethylene glycol
PLGA polylactic-coglycolic acid
PS polysorbate
PVA polyvinyl alcohol
PVP polyvinyl pyrrolidone

What is claimed is:

1. A liquid pharmaceutical formulation comprising an antibody, a buffer, and at least one oligopeptide, wherein (i) the oligopeptide comprises an arginine residue and consists of 2 to 10 amino acid residues but is not arg-asp, arg-glu, or poly-arginine, (ii) the buffer is acetate, glutamate, or phosphate and (iii) the pH of the formulation is about 4.5 to about 6.5.

2. The formulation of claim 1, wherein the antibody is present in a concentration of at least about:
a. 70 mg/mL,
b. 85 mg/mL,
c. 100 mg/mL,
d. 130 mg/mL,
e. 160 mg/mL
f. 200 mg/mL, or
g. 200 mg/mL to about 400 mg/mL.

3. The formulation of claim 1, wherein the antibody is selected from adalimumab, bevacizumab, blinatumomab, cetuximab, conatumumab, denosumab, eculizumab, erenumab, evolocumab, a PCSK9 binding polypeptide, infliximab, natalizumab, panitumumab, prezalumab, rilotumumab, rituximab, romosozumab, tezepelumab, and trastuzumab, or comprises the heavy chain and light chain sequences of an antibody selected from Table 2.

4. The formulation of claim 1, wherein the oligopeptide has a concentration of:
a. about 10 mM to about 500 mM or
b. about 100 mM to about 200 mM.

5. The formulation of claim 1, wherein the oligopeptide is an acetate salt.

6. The formulation of claim 1, wherein the oligopeptide is selected from Arg-Lys, Arg-Phe, Arg-Pro, Arg-Val, Arg-Ala, Asp-Arg, Lys-Arg, Pro-Arg, Leu-Arg, Val-Arg, Phe-Arg, Arg-Tyr, and Ala-Arg,.

7. The formulation of claim 1, further comprising (i) a second oligopeptide comprising arginine and consisting of two to 10 amino acid residues, wherein the second oligopeptide is a dipeptide comprising a basic, acidic, hydrophobic, or aromatic residue, (ii) one or more amino acids, or (iii) mixtures of any thereof.

8. The formulation of claim 8, wherein the second oligopeptide is selected from Arg-Lys, Arg-Pro, Arg-Ala, Asp-Arg, Lys-Arg, Pro-Arg, Val-Arg, Ala-Arg, Phe-Arg, and Arg-Tyr and the one or more amino acids are selected from arginine, proline, n-acetyl arginine, n-acetyl lysine, n-acetyl histidine, n-acetyl proline.

9. A method of reducing viscosity in a pharmaceutical formulation comprising an antibody, wherein the method comprises:
providing a solution at a pH of about 4.5 to about 6.5 comprising (i) the antibody, (ii) an oligopeptide salt, wherein the oligopeptide comprises an arginine residue, consists of 2 to 10 amino acid residues and is present in a viscosity-reducing concentration, and (iii) a buffer.

10. The method of claim 9, wherein the antibody is present in a concentration of at least about:
a. 70 mg/mL,
b. 85 mg/mL,
c. 100 mg/mL,
d. 130 mg/mL,
e. 160 mg/mL
f. 200 mg/mL, or
g. 200 mg/mL to about 400 mg/mL.

11. The method of claim 9, wherein the antibody is selected from adalimumab, bevacizumab, blinatumomab, cetuximab, conatumumab, denosumab, eculizumab, erenumab, evolocumab, a PCSK9 binding polypeptide, infliximab, natalizumab, panitumumab, rilotumumab, rituximab, romosozumab, tezepelumab, and trastuzumab, or comprises the heavy chain and light chain sequences of an antibody selected from Table 2.

12. The method of claim 9, wherein the oligopeptide has a concentration of:
a. about 10 mM to about 500 mM or
b. about 100 mM to about 200 mM.

13. The method of claim 9, wherein the oligopeptide is selected from Arg-Lys, Arg-Phe, Arg-Pro, Arg-Val, Arg-Ala, Asp-Arg, Lys-Arg, Pro-Arg, Leu-Arg, Val-Arg, Phe-Arg, Arg-Tyr, and Ala-Arg.

14. The method of claim 9, wherein the oligopeptide salt is an acetate salt.

15. The method of claim 9, wherein the oligopeptide salt is a lyophilized powder prior to being placed in solution.

16. The method of claim 9, wherein the solution further comprises (i) a second oligopeptide comprising arginine and consisting of two to 10 amino acid residues, wherein the second oligopeptide is a dipeptide comprising a basic, acidic, hydrophobic, or aromatic residue, (ii) one or more amino acids, or (iii) mixtures of any thereof.

17. The method of claim 16, wherein the second oligopeptide is selected from Arg-Lys, Arg-Phe, Arg-Pro, Arg-Val, Arg-Ala, Asp-Arg, Lys-Arg, Pro-Arg, Leu-Arg, Val-Arg, Ala-Arg, Phe-Arg, and Arg-Tyr and the one or more amino acids are selected from arginine, proline, n-acetyl arginine, n-acetyl lysine, n-acetyl histidine, n-acetyl proline or mixtures of any thereof.

18. The method of claim 9, wherein viscosity of the formulation is reduced by at least about 30% or at least about 50%.

19. A pharmaceutical formulation of tezepelumab, which comprises an excipient selected from arginine, proline, proline and arginine, N-acetyl arginine, Arg-Lys, Arg-Phe, Arg-Pro, Arg-Val, Arg-Ala, Asp-Arg, Lys-Arg, Pro-Arg, Leu-Arg, Val-Arg, or Ala-Arg.

20. A pharmaceutical formulation of PCSK9 binding polypeptide, wherein the formulation comprises an excipient selected from Arg-Phe, Arg-Ala, Arg-Val, Pro-Arg, Val-Arg, Ala-Arg, Phe-Arg, Arg-Tyr, and Arg-Phe.
